(19) United States
(12) Patent Application Publication
(10) Pub. No.: US 2025/0152682 A1
(43) Pub. Date: May 15, 2025

(54) LIQUID BOTULINUM TOXIN FORMULATION AND USE THEREOF

(71) Applicant: MERZ PHARMA GMBH & CO. KGAA, Frankfurt am Main (DE)

(72) Inventors: Karl-Heinz EISELE, Frankfurt am Main (DE); Klaus FINK, Frankfurt am Main (DE); Harold V. TAYLOR, Frankfurt am Main (DE); Claudia JATZKE, Frankfurt am Main (DE); Katja KÜHBACH, Frankfurt am Main (DE)

(73) Assignee: MERZ PHARMA GMBH & CO. KGAA, Frankfurt am Main (DE)

(21) Appl. No.: 18/838,555

(22) PCT Filed: Feb. 14, 2023
(86) PCT No.: PCT/EP2023/053647

§ 371 Date: Aug. 14, 2024

(30) Foreign Application Priority Data
Feb. 15, 2022 (EP) 22156843.9
Jul. 5, 2022 (EP) 22183172.0

(51) Int. Cl. A61K 38/48; A61K 8/64; A61K 8/66; A61K 38/38; A61K 47/02; A61Q 19/00

(52) U.S. Cl. CPC A61K 38/4893; A61K 8/64; A61K 8/66; A61K 38/385; A61K 47/02; A61Q 19/00; C12Y 304/24069

(57) ABSTRACT

The present invention relates to a liquid formulation comprising (i) botulinum toxin, (ii) human serum albumin (HSA), and optionally (iii) a tonicity agent and/or (iv) a buffering agent. In one aspect, the liquid formulation is characterized by a very low iron ion concentration. In another aspect, liquid formulation is characterized by the absence or very low concentration of tryptophan and/or N-acetyl-tryptophan. According to the present invention, the liquid formulation can be prepared by a method comprising the steps of contacting human serum albumin with a chelating agent to obtain a mixture and removing the chelating agent from the mixture. Furthermore, the present invention relates to the use of the liquid formulation in the treatment of therapeutic indications and cosmetic conditions.

### FIELD OF THE INVENTION

The present invention relates to a liquid formulation comprising (i) botulinum toxin, (ii) human serum albumin (HSA), and optionally (iii) a tonicity agent and/or (iv) a buffering agent. The liquid formulation is characterized by a very low iron concentration and/or the absence or very low concentration of tryptophan and/or N-acetyl-tryptophan. The present invention further relates to a liquid formulation prepared by a method comprising the steps of contacting human serum albumin with a chelating agent to obtain a mixture and removing the chelating agent from the mixture. Furthermore, the present invention relates to the use of the liquid formulation in the treatment of therapeutic indications and cosmetic conditions.

### BACKGROUND OF THE INVENTION

Botulinum neurotoxins (BoNTs; or botulinum toxin (BT)) are a family of bacterial neurotoxins that are widely used to treat a growing variety of neurologic, medical and cosmetic conditions. There are eight widely accepted “classical” BoNT serotypes designated BoNT/A-H). Two serotypes, type A (BoNT/A) and type B (BoNT/B), are currently in clinical use. BoNTs are produced by Clostridium spp., in particular Clostridium botulinum, in the form of high molecular weight (up to ˜900 kDa) complexes. These toxin complexes are composed of the active 150 kDa neurotoxin and several complexing proteins (non-toxic neurotoxin-associated proteins, NAPs).

The 150 kDa neurotoxin is synthesized as an inactive single chain polypeptide (˜150 kDa), which is proteolytically cleaved to give a light chain (LC, ˜50 kDa) and a heavy chain (HC, ˜100 kDa) connected by an inter-chain disulfide bond. The HC contains a C-terminal domain that mediates binding to receptors and an N-terminal domain that mediates translocation of the LC across endosomal membranes. The LC acts as a protease in neurons and cleaves neuronal SNARE proteins. This blocks the fusion of synaptic vesicles with the plasma membrane, thus inhibiting neurotransmitter release from selected neurons.

The formulation of BoNTs is very challenging, owing to their structural complexity and low product concentrations used. BoNT is highly susceptible to various conditions such as heat and alkaline pH. Thus, since BONT is only functional if its structure is intact, the challenge regarding the preparation of a medical dosage form of BONT is to formulate a composition that protects the BoNT from inactivation or partial loss of biological activity during production, storage or use of the product. Another problem is that pharmaceutical formulations of BoNT comprise extremely small quantities of the highly potent toxin (human lethal dose of about 0.1 to 1 ng/kg) in the range of only about 1 ng per vial. This aggravates the problem of loss of toxin activity due to surface denaturation. Furthermore, formulations of BoNTs are used for wide range of different applications and thus must be compatible with injections into very different types of tissue, such as muscles, different layers of skin (e.g., dermis, subcutis), or glands (e.g., salivary gland).

In view of the above, the main BoNT products currently available are provided as lyophilized powders, i.e., in a form that is stable over a long period of time when stored at 2-8° C. or even at room temperature (e.g., Xeomin®). Lyophilized forms of the BoNT/A complex were first introduced on the market in 1989 (Botox®, Allergan) and 1991 (Dysport®, Ipsen). In 2005, the first stable dosage form of the pure 150 kDa BoNT/A neurotoxin without complexing proteins was approved (Xeomin®; Merz Pharmaceuticals). However, these lyophilized products need to be reconstituted prior to use, a process potentially leading to dosing errors and sterility issues. Therefore, major efforts have been made to develop liquid formulations of BoNTs, which are more convenient to use and can be easily administered.

The first liquid formulation of BoNT was approved in 2000 and launched in Europe in 2001 as Neurobloc®. Neurobloc® (Eisai) is a sterile solution of the BoNT/B complex formulated in a buffer containing disodium succinate, sodium chloride, human serum albumin (HSA), sodium caprylate, and sodium N-acetyl-tryptophanate. However, this product may be painful when injected due to its acidic pH. For BoNT/A, only two liquid formulations have obtained market approvals so far: Innotox® (Medytox), but limited to Asian domestic markets (e.g., Korea, approval in 2013), and Alluzience® (Ipsen/Galderma) which was approved for use in Europe in 2021. Both these liquid formulations contain the BoNT/A toxin complex and, in addition to water, sodium chloride, a detergent (Innotox®: polysorbate 20; Alluzience®: polysorbate 80), an amino acid (Innotox®: methionine; Alluzience®: histidine) and an additional excipient (Innotox®: sodium phosphate as a buffer; Alluzience®: sucrose).

Despite these advances in the preparation of liquid botulinum toxin formulations, there is an ongoing need for stable liquid formulations of botulinum toxins, in particular there is still a need for new options to develop ready-to-use liquid formulations of botulinum toxin tat are stable during transport and storage.

### OBJECT OF THE INVENTION

It is therefore an object of the present invention to provide a stable liquid formulation of botulinum toxin that is suitable for use in cosmetic and therapeutic applications.

### SUMMARY OF THE INVENTION

The present invention is based on the surprising finding that human serum albumin (HSA) that has been pre-treated by incubating with a chelating agent, such as EDTA, followed by removing the chelating agent enables the preparation of a liquid formulation of botulinum toxin that not only exhibits an excellent storage stability but also a superior and improved light stability. Furthermore, the present invention is based on the surprising finding that iron ions, in particular Fe³⁺ ions, have a deleterious effect on the light stability of liquid botulinum toxin formulations containing HSA.

Furthermore, the present invention is based on the surprising finding that tryptophan and N-acetyl-tryptophan decrease the light stability of liquid botulinum toxin formulations containing human serum albumin (HSA). This finding is highly surprising since commercial HSA products, in addition to water and sodium chloride, usually contain sodium caprylate and N-acetyl-tryptophan to stabilize HSA at high temperatures, which in this way get into the botulinum toxin formulation. It is also surprising because tryptophan is described as stabilizing additive for botulinum toxin (see, e.g., EP 3 679 946).

Thus, in a first aspect, the present invention provides a liquid formulation, comprising
(i) botulinum toxin and
(ii) human serum albumin (HSA),
   wherein the liquid formulation contains Fe³⁺ ions in a concentration of less than 1 μM, preferably less than 500 nM, more preferably less than 250 nM, and most preferably less than 100 nM.

The liquid botulinum toxin formulation with low Fe³⁺ concentrations of, e.g., less than 1 μM, can be prepared by a method comprising step (a) of contacting human serum albumin with a chelating agent to obtain a mixture of human serum albumin and the chelating agent, and step (b) of removing the chelating agent from the mixture.

In a second aspect, the present invention provides a liquid formulation, comprising
(i) botulinum toxin and
(ii) human serum albumin (HSA),
   wherein the liquid formulation contains no tryptophan and N-acetyl-tryptophan or no more than 50 μM, preferably no more than 20 μM, more preferably no more than 10 UM, still more preferably no more than 1 μM, and most preferably 0 μM, of tryptophan and N-acetyl-tryptophan (total concentration of both Trp and N-AcTrp).

The liquid formulation according to the first and second aspect of the present invention preferably further comprises (iii) a tonicity agent, in particular sodium chloride, and (iv) a buffering agent, in particular a histidine buffer, phosphate buffer or a mixture thereof. The liquid botulinum toxin formulation free of or containing only very low concentrations of tryptophan and/or N-acetyl-tryptophan can be prepared by purifying the HSA starting material by a method such as dialysis and using the purified HSA material in the formulation of the liquid formulation of the present invention.

In a third aspect, the present invention provides a liquid formulation comprising:
(i) botulinum toxin and
(ii) human serum albumin (HSA),
   wherein the human serum albumin is prepared by a method comprising the following steps:
(a) contacting human serum albumin with a chelating agent to obtain a mixture of human serum albumin and the chelating agent, and
(b) removing the chelating agent from the mixture.

The contacting step (a) is preferably carried out by adding the chelating agent (e.g., EDTA) to a composition comprising HSA (e.g., a HSA solution) and incubating the resulting mixture for a predetermined time, optionally followed by dialysis against a buffer containing the chelating agent (e.g., EDTA). In step (b), the chelating agent is removed by any suitable method, for example by dialysis.

Furthermore, the liquid formulation according to the third aspect of the present invention preferably further comprises (iii) a tonicity agent, in particular sodium chloride, and/or (vi) a buffering agent, in particular a histidine buffer, phosphate buffer or a mixture thereof.

In a fourth aspect, the present invention relates to a method for preparing a liquid formulation according to the second aspect of the present invention, wherein the method comprises the following steps:
purifying a human serum albumin starting material to obtain a purified human serum albumin material, e.g., by subjecting the human serum albumin starting material to dialysis, diafiltration, ultrafiltration, ion exchange chromatography, affinity chromatography, hydrophobic interaction chromatography or field-flow fractionation, preferably dialysis,
mixing the obtained purified human serum albumin with botulinum toxin and optionally further components to obtain the liquid formulation,

An alternative method according to the fourth aspect of the invention comprises the following steps:
purifying a liquid composition comprising botulinum toxin and human serum albumin to obtain a purified liquid composition, e.g., by subjecting the liquid composition comprising botulinum toxin and human serum albumin to dialysis, diafiltration, ultrafiltration, ion exchange chromatography, affinity chromatography, hydrophobic interaction chromatography or field-flow fractionation, preferably dialysis,
mixing the obtained purified liquid composition with further components to obtain the liquid formulation.

In a fifth aspect, the present invention relates to a liquid formulation according to the present invention for use in therapy, especially for use in the treatment of neuromuscular diseases, pain, sialorrhea, hyperhidrosis, urological disorders, and neurological disorders.

In a sixth aspect, the present invention relates to the cosmetic (aesthetic) use of the liquid formulation of the present invention for the treatment of a cosmetic condition, preferably a skin condition, in particular for the treatment of skin lines or folds or wrinkles.

In a seventh aspect, the present invention provides a method of treating a disease or condition, comprising administering an effective amount of the liquid formulation of the present invention to a person in need thereof.

Preferred embodiments of the liquid formulation, its use and methods of use according to the present invention are set forth in the appended claims.

The present invention may be understood more readily by reference to the following detailed description of the invention and the example included therein.

### DETAILED DESCRIPTION OF THE INVENTION

The present invention is based on the unexpected finding that the use of a pre-treated human serum albumin (HSA) in the formulation of a liquid botulinum toxin formulation results in a reduced light sensitivity and, thus, in an improved light stability. Furthermore, it was unexpectedly found that very low amounts of iron ions (Fe³⁺ ions), achieved by chelating agent treatment followed by dialysis, drastically increase the light sensitivity of liquid botulinum toxin formulations containing human serum albumin (HSA). This is surprising since iron is relatively common and ubiquitous, and moreover, it was found that other metal ions such as copper, cobalt or nickel do not show this destabilizing effect.

Furthermore, the present invention is based on the unexpected finding that tryptophan and/or N-acetyl-tryptophan impair the light stability (photostability) of human serum albumin (HSA)-containing liquid botulinum toxin formulations. In particular, it was found that the removal of N-acetyl-tryptophan from conventional HSA products by chelating agent treatment and subsequent dialysis resulted in liquid HSA-containing formulations of botulinum toxin having an improved light stability.

The liquid formulation of the present invention advantageously provides an improved safety and dosing accuracy compared to lyophilized botulinum toxin preparations in powder form since the formulation does not need to be reconstituted prior to injection but is ready-to-use and can be used, e.g., in the form of a prefilled syringe. Further, the excellent stability of the liquid formulation facilitates and simplifies the transport and storage as well as the handling by the physician. In particular, the superior light stability of the liquid formulation of the present invention simplifies the manufacturing process, allows the product to be filled and packaged without extensive light protection, and reduces potential loss of activity due to light-exposed storage at the physician's place prior to use. In addition, the liquid formulation of the present invention does not contain substances which results in enhanced injection pain, thereby increasing acceptance of the formulation by physicians and patients, particularly in the aesthetic field.

Without being bound by theory, it is believed that the light sensitivity of the formulation of the present invention relates to substances such as N-acetyl-tryptophan and Fe³⁺ included in the HSA used for preparing the liquid botulinum toxin formulations. However, the exact mechanism of how the removal of these substances increases the ability of HSA to stabilize botulinum toxin against light remains unclear.

As used herein, the term “comprising”, like the terms “including” and containing”, and any variations thereof such as “comprises”, “includes” and “contains”, are intended to refer to a non-exclusive inclusion, such that a process, method, product-by-process, composition or formulation that comprises, includes, or contains an element or list of elements does not include only those elements but can include other elements not expressly listed for such process, method, product-by-process, composition or formulation. In addition, within the framework of the present invention, it is intended that each of the terms “comprise,”, “comprising”, “includes”, “including”, “contains”, “containing”, and any variations thereof, may be replaced by the term “consists” or “consisting”, or any variation thereof (e.g., “consists essentially of”), which will be understood to refer to an exclusive inclusion of the elements indicated.

The terms “a” and “an” and “the” and similar reference used in the context of the present invention are to be construed to cover both the singular and the plural and, thus, may also relate to “at least one” or “more than one”, unless otherwise indicated herein or clearly contradicted by the context.

The term “liquid formulation” or “liquid botulinum toxin formulation”, as used herein, generally refers to an aqueous formulation and is typically an aqueous solution. Herein, the term “liquid formulation” may be interchangeably used with “liquid composition”. The liquid formulation is generally a pharmaceutically acceptable liquid formulation or, in other words, a liquid pharmaceutical formulation. The liquid formulations of the present invention have typically a pH in the range of 5.0-8.0, particularly in the range of 5.5-7.5, and preferably in the range of 5.5-7.0 or 6.0-7.5, more preferably in the range of 6.0-7.0, and most preferably in the range of 6.0-6.5.

The term “pharmaceutically acceptable” as used herein, means that the liquid formulation does not cause unacceptable adverse side effects when administered to a human patient or subject. i.e., it means that the liquid formulation is suitable for human use. The aqueous solution may be a buffered solution, with or without a saline solution, and may be a physiological saline solution such as a buffered (e.g., phosphate and/or histidine buffered) physiological saline solution.

It is contemplated herein that the liquid formulation of the present invention can be stored in any suitable container system. A suitable container system for storing the liquid formulation of the present invention is any device having a partially or fully enclosed space that can be sealed or is sealed and can be used to contain, store, and/or transport liquid formulations. A container system is preferably a closed (or sealed) container made of, or partially or predominantly made of, glass or plastic (e.g., organic polymers) and includes, for example, containers in the form of (i) a syringe, (ii) a vial, (iii) a carpule, or (iv) an ampoule. In a preferred embodiment of the present invention, the liquid formulation is stored in a syringe in the form of a prefilled syringe, as known in the art.

The term “botulinum toxin”, as used herein, is not particularly limited and includes botulinum toxin of any serotype (e.g., BoNT/A-G). For example, the botulinum toxin may be of serotype A or B (BONT/A, BoNT/B). Preferably, the botulinum toxin is of serotype A, more preferably of serotype A1 (BoNT/A1), and most preferably BoNT/A1 produced by Clostridium botulinum Hall strain. In addition, the botulinum toxin may be a natural neurotoxin obtainable from the bacteria Clostridium botulinum or any other botulinum toxin such as a botulinum toxin obtainable from alternative sources, including recombinant technologies and genetic or chemical modification.

Furthermore, as used herein, the term “botulinum toxin” (“BT”) and the synonymously used term “botulinum neurotoxin” (“BONT”) are intended to refer to the pure botulinum neurotoxin and/or any complex thereof, i.e., any complex of the pure botulinum neurotoxin and complexing proteins (referred to as the “toxin complex”), unless otherwise stated, or the context otherwise indicates. Preferably, within the framework of the present invention, the botulinum toxin is a botulinum neurotoxin free of complexing proteins, more preferably a botulinum neurotoxin free of complexing proteins of serotype A.

The term “pure botulinum neurotoxin”, as used herein, means the botulinum neurotoxin free of complexing proteins (sometimes also referred to as the “neurotoxic component”), or more precisely, the botulinum neurotoxin without neurotoxin-associated complexing proteins (NAPs). The pure botulinum neurotoxin is the (active) neurotoxic polypeptide that ultimately inhibits acetylcholine release. It is a di-chain protein comprised of a light chain (LC; about 50 kDa) and a heavy chain (HC; about 100 kDa), held together by a disulfide bond. The active neurotoxic polypeptide may therefore also be referred to herein as the “150 kDa neurotoxin”, “Clostridium botulinum neurotoxin (150 kD)” or “neurotoxic component”.

The term “toxin complex”, as used herein, refers to a high-molecular complex of the neurotoxic component and a set of complexing proteins (NAPs). In particular, the term “toxin complex” includes the 900 kDa, 500 kDa, and 300 kDa C. botulinum type A toxin complexes. The complexing proteins are nontoxic nonhaemagglutinin (NTNHA) and, in strains of serotypes A-D, different haemagglutinins (HAs). For example, the 900 kDa complex is included in onabotulinumtoxin A (Botox®/Vistabel®, Allergan, Inc., Irvine, CA, USA). Also, a toxin complex as active agent is contained in Dysport® (Azzalure®, Ipsen, Paris, France), Alluzience® (Ipsen/Galderma) and Innotox® (Medytox). This is, the toxin complexes contained in Botox®, Dysport®, Alluzience® and Innotox® are toxin complexes within the meaning of the present invention and, thus, a “botulinum toxin” according to the present invention. In accordance with the present invention, the botulinum toxin may be present in the liquid formulation of the present invention in a range of 1-1000 U/ml, preferably in the range of 10-200 U/ml, more preferably in the range of 20-150 U/ml, and most preferably in the range of 50 U/ml to 100 U/ml.

The term “units” or “U”, as used herein refers to the biological activity (biological potency) of the toxin and relates to the dose that is lethal to 50% of mice tested (LD₅₀). More specifically, within the context of the present invention, the LD₅₀ is measured using the mouse bioassay (MBA), unless otherwise stated. The MBA determines the mean lethal dose (LD₅₀) of toxin/neurotoxin after intraperitoneal injection in mice, i.e., the dose of toxin/neurotoxin capable of killing 50% of a group of mice. On this basis, 1 unit (U) of toxin/neurotoxin, as used herein, is defined as one mouse LD₅₀ (1.0 LD₅₀=1.0 U).

The LD₅₀ mouse bioassay is the gold standard among various biological, chemical or immunological detection methods for botulinum toxin and is known to those skilled in the art (see, e.g., Pearce, L. B.; Borodic, G. E.; First, E. R.; MacCallum, R. D. Measurement of botulinum toxin activity: Evaluation of the lethality assay. Toxicol. Appl. Pharmacol. 1994, 128:69-77). A person skilled in the art will be able to determine suitable botulinum toxin concentrations depending on the serotype and the intended use. Alternatively, also a cell-based assay can be used to determine botulinum toxin activity, as described in WO 2009/114748, WO 2013/049508 or WO2014/207109. A person skilled in the art will be able to correlate botulinum toxin activity results obtained with a cell-based assay with results obtained in the mouse LD₅₀ assay by calibration using a LD₅₀ reference standard.

Due to differences in the LD₅₀ tests used by manufacturers of commercial botulinum toxin formulations, the unit potencies indicated by the manufacturers for their commercial botulinum toxin formulations is proprietary and cannot easily be compared. Therefore, within the framework of the present invention, the conversion rates provided below are used to establish the comparative potencies of incobotulinumtoxinA (“INCO”; Xeomin®, Bocouture®; botulinum toxin serotype A, free of complexing proteins; Merz Pharmaceuticals GmbH), onabotulinumtoxinA (“ONA”; Botox®, Vistabel®; botulinum toxin complex of serotype A; Allergan Inc.), abobotulinumtoxinA (“ABO”; Dysport®, Azzalure®; botulinum toxin complex of serotype A; Medicis Pharmaceutical Corp., Galderma Lab.), rimabotulinumtoxinB (“RIM”; Myobloc®, NeuroBloc®; botulinum toxin serotype B; Solstice Neurosciences Inc.), and PurTox® (“TBD”; botulinum toxin serotype A; Mentor Worldwide LLC). For use herein, the conversion rate of ONA and INCO is 1:1. The conversion rate of ONA/INCO: ABO is 1:2.5. The conversion rate of ONA/INCO: RIM is 1:50, and the conversion rate of ONA/INCO: TBD is 1:1.5. Furthermore, within the context of the present invention, 1 U of INCO (Xeomin®) and 1 U of onabotulinumtoxinA (“ONA”; Botox®) shall be deemed to correspond to one mouse LD50 (1.0 LD50), or 1 U, measured as described above.

The term “human serum albumin” or its abbreviation “HSA”, as used herein, is intended to refer to donor HSA (HSA derived from human blood or, more precisely, from human plasma) and recombinant HSA. Preferably, the human serum albumin is donor HSA. Within the present invention, the human serum albumin (HSA) acts as a stabilizing protein. As used herein, the term “stabilizing protein” generally refers to a polypeptide that results in an increased stability of the botulinum toxin. In accordance with the present invention, the HSA may be present in the liquid formulations in an amount of 0.001-2.0% w/v, preferably in an amount of 0.001-1.00% w/v, more preferably in amount of 0.01-0.5% w/v, still more preferably in an amount of 0.02-0.3% w/v, and most preferably in an amount of 0.03-0.15% w/V.

In a first aspect, the present invention relates to a liquid formulation, comprising
(i) botulinum toxin and
(ii) human serum albumin,
   wherein the liquid formulation contains Fe³⁺ ions in a concentration of less than 1 μM.

Preferably, the Fe³⁺ concentration in the liquid formulation is less than 1000 nM, less than 750 nM, less than 500 nM, or less than 250 nM, more preferably, the Fe³⁺ concentration in the liquid formulation is less than 200 nM, less than 150 nM, or less than 100 nM, particularly preferable the Fe³⁺ concentration in the liquid formulation is less than 50 nM or less than 10 nM, and most preferably the Fe³⁺ concentration in the liquid formulation is less than 1 nM, less than 100 pM, less than 10 pM, or less than 1 pM.

The concentration of metal ions (e.g., Ca²⁺, Co²⁺, Cu²⁺, Ni²⁺, or Fe³⁺) can be determined by measuring methods known to those skilled in the art such as atomic absorption spectroscopy (AAS), inductively coupled plasma mass spectrometry (ICP-MS), and inductively coupled plasma atomic emission spectroscopy (ICP-AES) (also referred to as inductively coupled plasma optical emission spectrometry (ICP-OES)). Preferably, ICP-MS or ICP-OES, in particular ICP-OES, may be used for measuring the concentration of metal ions (see also Second Supplement to USP38-NF 33, Chemical Tests/<233> Elemental Impurities-Procedure 1 (ICP-OES) and Procedure 2 (ICP-MS), 2015).

Without being limited by theory, it is believed that the HSA material used for preparing the liquid formulation of the present invention, in particular the donor HSA derived from human blood that contains large amounts of iron ions, contains a significant amount of Fe³⁺ ions which are responsible—at least in part—for the photosensitivity-inducing properties of unpurified (untreated) HSA.

In addition to the advantages mentioned above, this aspect of the present invention is also advantageous in the development of manufacturing processes and new formulations. For example, equipment (e.g., vessels) and materials (e.g., excipients) can be selected in such a way that as little iron as possible is present in the end product.

The liquid botulinum toxin formulation of the present invention with low Fe³⁺ concentrations may be prepared by a method comprising step (a) of contacting human serum albumin with a chelating agent to obtain a mixture of human serum albumin and the chelating agent, and step (b) of removing the chelating agent from the mixture.

Preferably, the method for preparing the liquid formulation according to the first aspect of the present invention is the same as the method described in connection with the third aspect of the present invention. Furthermore, the liquid formulation according to the first aspect of the present invention may have the same composition as the composition of the liquid formulation according to the third aspect of the present invention. Therefore, all explanations, comments, disclosures, definitions and the like given for the liquid formulation according to the third aspect of the present invention equally apply to the liquid formulation according to the first aspect of the present invention, unless explicitly stated otherwise.

In a second aspect, the present invention relates to a liquid formulation, comprising
(i) botulinum toxin and
(ii) human serum albumin,
   wherein the liquid formulation contains no or no more than 50 μM tryptophan and N-acetyl-tryptophan.

The term “contains no” tryptophan and N-acetyl-tryptophan, as used herein, means that the liquid formulation is free of, or completely free of, tryptophan and N-acetyl-tryptophan. Specifically, it means that the formulation contains no added tryptophan and N-acetyl-tryptophan. Alternatively, it means that the concentration of tryptophan and N-acetyl-tryptophan in the liquid formulation is 0 μM (i.e., according to general rules of rounding <0.5 μM), in particular ≤0.1 μM or ≤0.01 μM or ≤0.001 μM, more particularly 0 nM (i.e., according to general rules of rounding <0.5 nM. The term “contains no more than” of tryptophan and N-acetyl-tryptophan, as used herein, means that the total amount of tryptophan and N-acetyl-tryptophan may be ≤50 M, and is preferably ≤20 μM, more preferably ≤10 μM, still more preferably ≤1 μM, yet more preferably ≤0.1 μM, and most preferably ≤0.01 μM or ≤0.001 μM. Thus, the term “contains no or no more than”, as used herein, means that the total amount of tryptophan and N-acetyl-tryptophan in the liquid formulation is between 0 μM and X or between 0 nM and X, with X being 50 μM, 20 μM, 10 μM, 1 μM, 0.1 μM, 0.01 μM and 0.001 μM.

The concentration of the amino acids tryptophan and N-acetyl-tryptophan can be determined by various techniques (e.g., DC, HPLC, LC-MS, GC-MS, CE etc.), as known to those skilled in the art. For example, N-acetyl-tryptophane can be determined by separation using liquid chromatography on a reversed-phase column and UV detection at 280 nm as described in Nelis et al. (Nelis et al., J. Chromatogr., 1985, 333 (2): 381-387) or by a method based on UV spectrophotometry of the acid-soluble fraction remaining after precipitation of the protein as described in Yu and Finlayson (Yu, M. W. and Finlayson, J. S., J. Pharm. Sci., 1984, 73 (1): 82-86). Tryptophane can, e.g., be quantitatively determined using a method involving liquid chromatography-tandem mass spectrometry as described by Wentao et al. (Wentao et al., Analytical and Bioanalytical Chemistry, 2011, 401:3249-3261).

The term “tryptophan”, as used herein, refers to L-tryptophan, D-tryptophan or a mixture of L-tryptophan and D-tryptophan (D/L-tryptophan). Likewise, the term “N-acetyl-tryptophan”, as used herein, refers to N-acetyl-L-tryptophan, N-acetyl-D-tryptophan or a mixture of N-acetyl-L-tryptophan and N-acetyl-D-tryptophan (N-acetyl-D/L-tryptophan).

The human serum albumin used for preparing the liquid formulation is preferably a human serum albumin material containing no more than 50 mM, more preferably no more than 20 mM or 10 mM, still more preferably no more than 1 mM or 0.1 mM, and most preferably no more than 0.01 nM or 0 mM tryptophan and N-acetyl-tryptophan.

It is pointed out that commercial donor HSA products all contain N-acetyl-tryptophan in significant amounts (>10 mM). Thus, such products need to be purified to lower the amount of N-acetyl-tryptophan to the desired level prior to use for formulation of the liquid formulation of the present invention. Suitable methods for preparing the “purified HSA” are described in detail below in connection with the fourth aspect of the present invention. In brief, human serum albumin with no, or a low content of, tryptophan and N-acetyl-tryptophan (also referred to herein as “purified HSA”) can be obtained by subjecting a human serum albumin starting material to dialysis, diafiltration, ultrafiltration, ion exchange chromatography, affinity chromatography, hydrophobic interaction chromatography, field flow fractionation or precipitation (e.g., salt precipitation, ethanol precipitation), resulting in the removal of tryptophan and/or N-acetyl-tryptophan.

In accordance with the present invention, the liquid formulation of the present invention, e.g., the liquid formulation according to the first, second and/or third aspect of the present invention, may further comprise one or both of (iii) a tonicity agent and (iv) a buffering agent as described in more detail herein below, in particular in connection with the liquid formulation according to the third aspect of the present invention.

Furthermore, it is also contemplated herein that the liquid formulation in accordance with the second aspect of the present invention preferably contains Fe³⁺ ions in a concentration of less than 1000 nM. Specifically, the Fe³⁺ concentration in the liquid formulation is preferably less than 750 nM, less than 500 nM, or less than 250 nM, more preferably, the Fe³⁺ concentration in the liquid formulation is less than 200 nM, less than 150 nM, or less than 100 nM, particularly preferable the Fe³⁺ concentration in the liquid formulation is less than 50 nM or less than 10 nM, and most preferably the Fe³⁺ concentration in the liquid formulation is less than 1 nM, less than 100 pM, less than 10 pM, or less than 1 pM.

As mentioned above, the Fe³⁺ ions have also been found to decrease the light stability of botulinum toxin and, thus, add to the destabilizing effect of tryptophan and N-acetyl-tryptophan. It is believed that the HSA material used for preparing the liquid formulation of the present invention, in particular the donor HSA derived from human blood, contains a significant amount of Fe³⁺ ions which contributes to the light stability decreasing properties of unpurified (untreated) HSA. To achieve a Fe³⁺ concentration as low as possible, the equipment (e.g., vessels) and materials (e.g., excipients) should be selected in such a way that as little iron as possible is present in the end product by, e.g., selecting vessels made of non-iron containing materials such as polypropylene or polycarbonate.

The concentration of metal ions (e.g., Fe³⁺ and Ca²⁺, Co²⁺, Cu²⁺, Ni²⁺) can be determined by measuring methods known to those skilled in the art and disclosed above.

The low Fe³⁺ content of the liquid botulinum toxin formulation of the present invention can be achieved by a method described in detail in connection with the fourth aspect of the present invention, in particular by a method comprising contacting human serum albumin with a chelating agent, followed by removing the chelating agent as described herein.

In accordance with the present invention, the liquid formulation may further comprise one or more additional pharmaceutically acceptable excipients, unless otherwise stated or intended. For example, the liquid formulation may contain one or more of glycerol, sucrose, lactose, mannitol, dextran, hyaluronic acid, polyvinylpyrrolidone, lactic acid, citric acid, amino acid(s) (other than tryptophan and N-acetyl-tryptophan), benzyl alcohol, lidocaine, gelatine, hydroxyethyl starch (HES), polyethylene oxide, and polysorbate (e.g., polysorbate 20 and polysorbate 80). Other suitable pharmaceutically acceptable excipients comprise those well known in the art, see, e.g., Remington's Pharmaceutical Sciences, Mack Publishing Company, Easton, Pennsylvania.

It is, however, also contemplated herein that the liquid formulation of the present invention specifically lacks certain components (i.e., compounds, materials or substances), for example chelating agents and/or phosphate, detergents, polysaccharides, amino acids, stabilizing peptides, and the like, including any combination thereof. As used herein, the term “detergent”, is synonymously used with “surfactant” and is intended to include non-ionic and ionic detergents. The term “stabilizing peptide”, as used herein, generally means a peptide consisting of 5 to 50 amino acids, such as a peptide of 10 to 40 amino acids or 15 to 30 amino acids. Hence, the term “stabilizing peptide” excludes HSA.

In one embodiment, the liquid formulation of the present invention does not contain detergents, particularly does not contain polysorbate, more particularly does not contain polysorbate 20 and/or polysorbate 80. In another embodiment, the liquid formulation of the present invention does not contain alginate. In another embodiment, the liquid formulation of the present invention does not contain succinate. In another embodiment, the liquid formulation of the present invention does not contain one or more (e.g., 2, 3, 4 or 5) amino acids selected from the group consisting of: arginine, glutamic acid, methionine, tryptophan, and serine. In another embodiment, the liquid formulation of the present invention does not contain a saccharide, such as a mono-, oligo- or polysaccharide or a mixture thereof. In particular, the liquid formulation of the present invention may not contain one or more (e.g., 2, 3 or 4) of sucrose, lactose, maltose, and trehalose. In another embodiment, the liquid formulation of the present invention does not contain a chelating agent, in particular those described herein in connection with the present invention, and/or phosphate. It is also contemplated within the present invention that the liquid formulation lacks more than one, or all of the compounds mentioned above.

In one embodiment, the liquid formulation of the present invention lacks (i) detergents and mono-, oligo- and polysaccharides, (ii) detergents and any amino acid, or detergents and all amino acids except histidine, (iii) detergents and stabilizing peptides, (iv) mono-, oligo- and polysaccharides and any amino acid, or mono-, oligo- and polysaccharides and all amino acids except histidine, (v) mono-, oligo- and polysaccharides and stabilizing peptides, (vi) any amino acids and stabilizing peptides, or all amino acids except histidine and stabilizing peptides, (vii) detergents, mono-, oligo- and polysaccharides and any amino acid, or detergents, mono-, oligo- and polysaccharides and all amino acids except histidine, (viii) detergents, mono-, oligo- and polysaccharides, and stabilizing peptides, (ix) detergents, any amino acid, and stabilizing peptides, or detergents, all amino acids except histidine, and stabilizing peptides, (x) mono-, oligo- and polysaccharides, any amino acid, and stabilizing peptides, or mono-, oligo- and polysaccharides, all amino acids except histidine, and stabilizing peptides, (xi) detergents, mono-, oligo- and polysaccharides, any amino acid, and stabilizing peptides, or detergents, mono-, oligo- and polysaccharides, all amino acids except histidine, and stabilizing peptides.

In another embodiment, the liquid formulation of the present invention does not contain any other amino acid than histidine. In another embodiment, the liquid formulation of the present invention does not contain any mono-, di- and trisaccharides. In another embodiment, the liquid formulation of the present invention does not contain any other stabilizing peptide or protein than HSA. In another embodiment, the liquid formulation of the present invention does not contain phosphate such as in the form of a phosphate buffer.

In yet other embodiments, the liquid formulation of the present invention lacks (i) succinate and a detergent (e.g., polysorbate), (ii) succinate and methionine, (iii) succinate and sucrose, (iv) a detergent (e.g., polysorbate) and methionine, (v) a detergent (e.g., polysorbate) and sucrose, (vi) methionine and sucrose, (vii) succinate, a detergent (e.g., polysorbate) and methionine, (xiii) succinate, a detergent (e.g., polysorbate) and sucrose, (ix) succinate, methionine and sucrose, (x) a detergent (e.g., polysorbate), methionine and sucrose, and (xi) succinate, a detergent (e.g., polysorbate), methionine and sucrose, (xii) a detergent (e.g. polysorbate) and histidine, (xiii) a detergent (e.g. polysorbate), histidine and sucrose).

Furthermore, any of the liquid formulations of the present invention that lacks one or more components (i.e., compounds, materials or substances) may further lack a chelating agent, in particular the chelating agents described herein (e.g., EDTA), and/or phosphate.

Further, a preferred liquid formulation of the present invention comprises (i) botulinum toxin, (ii) HSA, (iii) a tonicity agent, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.01% w/v to 1.0% w/V, (iii) a tonicity agent, preferably sodium chloride, in an amount of 0.01% w/v to 2.0% w/v, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.01% w/v to 0.5% w/V, (iii) a tonicity agent, preferably sodium chloride, in an amount of 0.1% w/v to 1.5% w/v, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.05% w/v to 0.25% w/V, (iii) a tonicity agent, preferably sodium chloride, in an amount of 0.6% w/v to 1.2% w/v, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.01% w/v to 0.5% w/V, (iii) sodium chloride as a tonicity agent in an amount of 0.9% w/v, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.05% w/v to 0.25% w/V, (iii) sodium chloride as a tonicity agent in an amount of 0.9% w/v, and no or no more than 50 μM tryptophan and N-acetyl-tryptophan.

It is also contemplated herein that in the preferred and particularly preferred liquid formulations described in the preceding and following paragraph the upper limit of the concentration of tryptophan and N-acetyl-tryptophan is below 50 μM, preferably 10 μM, more preferably 1 μM, still more preferably 0.1 μM, and most preferably 0.01 μM or 0.001 μM. In addition, it is further contemplated herein that the preferred and particularly preferred liquid formulations described in the preceding and following paragraph may be characterized by a concentration of Fe³⁺ ions of less than 1000 nM or less than 500 nM, more preferably less than 250 nM or less than 100 nM, and most preferably less than 10 nM or less than 1 nM.

A preferred liquid formulation of the present invention comprises (i) botulinum toxin, (ii) HSA, (iii) a tonicity agent, (iv) a buffering agent, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.01% w/v to 1.0% w/V, (iii) a tonicity agent, preferably sodium chloride, in an amount of 0.01% w/v to 2.0% w/v, (iv) a buffering agent in a concentration of 1 mM to 100 mM, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.01% w/v to 0.5% w/v, (iii) a tonicity agent, preferably sodium chloride, in an amount of 0.01% w/v to 2.0% w/v, (iv) a buffering agent in a concentration of 1 mM to 100 mM, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.05% w/v to 0.25% w/V, (iii) a tonicity agent, preferably sodium chloride, in an amount of 0.1% w/v to 2.0% w/V, (iv) a buffering agent in a concentration of 1 mM to 100 mM, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.05% w/v to 0.25% w/V, (iii) a tonicity agent, preferably sodium chloride, in an amount of 0.6% w/v to 1.3% w/v, (iv) a buffering agent in a concentration of 2 mM to 50 mM, and no or no more than 50 μM tryptophan and N-acetyl-tryptophan. A preferred liquid formulation of the present invention comprises (i) botulinum toxin at a concentration of 10 U/ml to 200 U/ml, (ii) HSA in an amount of 0.05% w/v to 0.25% w/V, (iii) sodium chloride in an amount of 0.9% w/v, (iv) a buffering agent in a concentration of 5 mM to 20 mM, and no or no more than 50 μM tryptophan and N-acetyl-tryptophan.

A preferred liquid formulation of the present invention comprises (i) botulinum toxin, (ii) HSA, (iii) sodium chloride as a tonicity agent, (iv) a buffering agent selected from histidine, phosphate and a mixture thereof as a buffering agent, wherein the buffering agent is preferably histidine, and no or no more than 50 μM tryptophan and N-acetyl-tryptophan.

A particularly preferred liquid formulation comprises (i) botulinum toxin at a concentration of 10 U/ml to 200 U/ml, (ii) HSA in an amount of 0.01% w/v to 1.0% w/V, (iii) sodium chloride in an amount of 0.01% w/v to 2.0% w/v, preferably 0.9% w/V, (iv) a buffering agent selected from histidine, phosphate and a mixture thereof at a concentration of 1 mM to 100 mM, wherein the buffering agent is preferably histidine at a concentration of 1 mM to 100 mM, and no or no more than 50 μM tryptophan and N-acetyl-tryptophan.

Another particularly preferred liquid formulation of the present invention comprises (i) botulinum toxin at a concentration of 10 U/ml to 200 U/ml, (ii) HSA in an amount of 0.01% w/v to 0.5% w/V, (iii) sodium chloride in an amount of 0.9% w/V, (iv) a buffering agent selected from histidine, phosphate and a mixture thereof at a concentration of 1 mM to 100 mM, wherein the buffering agent is preferably histidine at a concentration of 1 mM to 100 mM, and no or no more than 50 μM tryptophan and N-acetyl-tryptophan.

Yet another particularly preferred liquid formulation of the present invention comprises (i) botulinum toxin at a concentration of 10 U/ml to 200 U/ml, (ii) HSA in an amount of 0.05% w/v to 0.25% w/V, (iii) sodium chloride in an amount of 0.9% w/v, (iv) a buffering agent selected from histidine, phosphate and a mixture thereof at a concentration of 1 mM to 100 mM, preferably at a concentration of 2 mM to 50 mM or, more preferably 5 mM to 20 mM, wherein the buffering agent is preferably histidine at a concentration of 1 mM to 100 mM, preferably at a concentration of 2 mM to 50 mM, more preferably 5 mM to 20 mM, and no or no more than 50 μM tryptophan and N-acetyl-tryptophan.

Furthermore, the preferred and particularly preferred liquid formulations describe above have a pH that is preferably in the range of 6.0 to 7.5, more preferably in the range of 6.5 to 7.0. Also, the botulinum toxin is preferably of serotype A and, more preferably, is the neurotoxic component of serotype A.

A preferred exemplary liquid formulation of the present invention is the following:

### Formulation 1:

50 U/ml BoNT/A
0.9% NaCl (9 mg/mL)
0.085% HSA (purified, e.g., dialyzed) (0.85 mg/mL)
0.155% histidine (1.55 mg/ml; ˜10 mM)
Trp and N-AcTrp <50 μm
pH 6.0.

The purified HSA of exemplary Formulation 1 can be prepared by a process as described in detail in connection with the method according to the fourth aspect of the present invention.

In accordance with the present invention, the liquid formulation of the present invention is preferably prepared, or obtainable, by the method according to the fourth aspect of the present invention. This method allows for the removal of substances included in the HSA starting material (e.g., commercial HSA products), including N-acetyl-tryptophan, which deteriorate the light stability of the liquid botulinum toxin formulation.

In a third aspect, the present invention relates to a liquid formulation comprising
(i) botulinum toxin and
(ii) human serum albumin (HSA),
   wherein the liquid formulation is prepared by a method comprising the following steps:
(a) contacting human serum albumin with a chelating agent to obtain a mixture of human serum albumin and the chelating agent, and
(b) removing the chelating agent from the mixture.

As regards component (i) of the liquid formulation of the present invention, the botulinum toxin is not particularly limited and includes botulinum toxin of any serotype (e.g., BONT/A-G). Preferably, the botulinum toxin is as defined hereinabove.

As regards component (ii) of the liquid formulation, the “human serum albumin” or “HSA” is intended to refer to donor HSA and recombinant HSA, and is preferably donor HSA, as defined hereinabove. The HSA may be present in the liquid formulation in an amount of 0.001-2.0% w/v, preferably in an amount of 0.001-1.00% w/v, more preferably in amount of 0.01-0.5% w/v, still more preferably in an amount of 0.02-0.3% w/v, and most preferably in an amount of 0.03-0.15% w/v.

In accordance with the present invention, the liquid formulation of the present invention is prepared by a method comprising at least steps (a) and (b), with the first step being as follows:
(a) contacting human serum albumin with a chelating agent to obtain a mixture of human serum albumin and the chelating agent.

As used herein, the term “contacting” is intended to be broadly interpreted as bringing two or more components together. This can be achieved by a variety of different methods, such as dissolving, mixing, suspending, blending, slurrying, stirring, flowing by, adsorbing, binding, extracting etc. Typically, the chelating agent is added to a composition comprising HSA (e.g., a solution of HSA), followed by mixing to obtain a homogeneous mixture, and optionally further processing steps (e.g., dialysis against a chelating agent containing buffer).

Accordingly, it is also contemplated that “contacting” can be effected by loading the HSA material, e.g., in the form of an aqueous solution, onto an immobilized metal affinity resin (IMAC). The term “immobilized metal affinity resin” (IMAC), as used herein, includes, but is not limited to, resins containing an immobilized functional moiety (e.g., iminodiacetic acid) capable of binding and coordinating multivalent cations, for example Chelating-Sepharose, Fractogel-EMD-Chelate, POROS 20MC, Matrex Cellufine Chelate, TALON, and Chelex 100 resin. Such immobilized metal affinity resins are commonly used in the form of chromatographic metal affinity columns as known to those skilled in the art. Furthermore, as used herein, the term “contacting” also includes batch mode binding using a suitable immobilized metal affinity resin.

As regards step (a), it is further pointed out that the terms “human serum albumin” and “chelating agent”, as used in step (a) of the method for preparing the liquid formulation of the present invention, are not intended to imply any restrictions as to their physical form or to exclude the presence of other substances or compounds that are mixed with, or included in, the HSA and the chelating agent. This means that the “human serum albumin” that is contacted in step (a) with the chelating agent may be present in any form, such as in the form of a solid or a liquid (e.g., an aqueous composition or aqueous solution). Likewise, the “chelating agent” that is contacted in step (a) with human serum albumin may be present in any form, such as in the form of a solid or a liquid (e.g., an aqueous composition or aqueous solution). Moreover, the expression “contacting human serum albumin with a chelating agent” does not exclude that (i) the human serum albumin is in the form of a composition (e.g., a solid composition or liquid composition, in particular an aqueous composition or aqueous solution) that contains one or more additional components included in the final liquid formulation, such as a tonicity agent or a buffering agent or, and/or (ii) the chelating agent is in the form of a composition (e.g., a solid composition or liquid composition, in particular an aqueous composition or aqueous solution) that contains one or more additional components included in the final liquid formulation, such as a tonicity agent or a buffering agent.

Preferably, the mixture obtained in step (a) is an aqueous mixture. This aqueous mixture can be prepared by different ways. For example, the human serum albumin may be in the form of an aqueous composition, e.g., an aqueous solution, which is mixed with the chelating agent that may be present in solid form or in liquid form, e.g., in the form of an aqueous solution of the chelating agent. Furthermore, the human serum albumin may be in the form of a solid, e.g., a lyophilized material, which is mixed with a chelating agent and an aqueous solution or with an aqueous solution of the chelating agent. Preferably, the human serum albumin is in the form of an aqueous composition, more preferably an aqueous solution, and the chelating agent is a solid or an aqueous composition (e.g., aqueous solution).

In particular, the HSA that is contacted in step (a) with a chelating agent may be in the form of an aqueous solution containing at least 5% w/v HSA, more preferably 10-30% w/v HSA, and most preferably 20% w/v HSA. Furthermore, the pH of the mixture obtained in step (a) may be adjusted to a pH in the range of 6.0-9.0, preferably 6.5-8.5, more preferably 7.0-8.5, and most preferably 7.0-8.0.

Moreover, the contacting step (a) of the method for preparing the liquid formulation according to the present invention may comprise several sub-steps. For example, in one embodiment, contacting step (a) comprises, or consists (only) of, the steps of mixing a chelating agent (e.g., EDTA) and human serum albumin, incubating said mixture for a determined time, and optionally subjecting said mixture to dialysis against a buffer containing a chelating agent, the chelating agent being preferably the same as that used in the incubating sub-step. In another embodiment, said mixture is not incubated but (directly) subjected to dialysis against a buffer containing a chelating agent, the chelating agent being preferably the same as that used in the step of mixing a chelating agent (e.g., EDTA) and human serum albumin. In another embodiment, the chelating agent and human serum albumin are not mixed prior to dialysis. This is, contacting step (a) may comprise, or consist (only) of, the step of subjecting said mixture to dialysis against a buffer containing a chelating agent.

Preferably, step (a) comprises, or consists (only) of, a step of adding a chelating agent to a composition comprising HSA (e.g., a solution of HSA) or of mixing a chelating agent with a composition comprising HSA (e.g. a solution of HSA). The resulting mixture is then incubated for some time, e.g., allowed to stand for a given time without stirring or stirred for a given time.

The time of incubation is not confined to a specific range but is typically at least 0.5 hours, particularly at least 1 hour, and more particularly at least 2 hours. The upper limit of the time of incubation is not critical and may be, e.g., 1 hour, 2 hours, 5 hours or 10 hours. Hence, the incubation time may be, for examples, 0.5 to 5 hours or 1 hour to 10 hours. Likewise, the incubation temperature is not particularly limited and may, for example, be within the range of 0° C. to 60° C. Preferably, the temperature is from 0° C. to 30° C. This means that room temperature (20° C. or 25° C.) is a suitable temperature within the present invention. As known to the skilled person, the temperature influences the reaction time. Generally, the incubation conditions (e.g., time and temperature) are selected such that the residual amount of chelating agent (e.g., EDTA) that is contained in the final product is 100 μM or less, preferably 10 μM or less, more preferably 1 μM or less.

Optionally, the incubation step is followed by further processing such as dialysis of the incubated mixture against a buffer containing a chelating agent, typically the same chelating agent as that used in the incubation step. The chelating agent used in this optional dialyzing step is preferably contained in the dialysis buffer at a concentration of 0.1 mM-1000 mM, more preferably 1 mM-200 mM, and most preferably 10 mM-100 mM. It is also preferred that the buffer used in the dialyzing step (i) has a pH of 7.5-8.5, (ii) further comprises a buffering agent, preferably according to the final composition, or (iii) further comprises a tonicity agent, preferably 0.9% sodium chloride w/v, or (i) and (ii), or (i) and (iii), or (ii) and (iii), or (i) and (ii) and (iii).

Within the present invention, the term “chelating agent”, as used herein, is not particularly limited as far as it is capable of binding metal ions. The term “chelating agent”, as used herein, may also be referred to “chelator” or “sequestering agent”. The chelating agents for use herein are typically metal ion-binding organic compounds. The metal ions generally form multiple coordinate bonds with the organic chelating agents which act as polydentate ligands.

Suitable chelating agents for use herein include, but are not limited to, aminopolycarboxylic acids (e.g., aminopolycarboxylic acids having three to six, preferably four, carboxylic acid functional groups) and other compounds such as citrate, porphyrins, TPEN (N,N,N′,N′-tetrakis(2-pyridinylmethyl)-1,2-ethanediamine), TETA (triethylenetetramine), and mixtures thereof. Exemplary aminopolycarboxylic acids include NTA (nitrilotriacetate), DOTA (1,4,7,10-tetraazacyclododecan-1,4,7,10-tetraacetate), TED (ethylenediaminotriacetate), EDTA (ethylenediaminetetraacetic acid), EGTA (ethylene glycol-bis(β-aminoethylether)-N,N,N′,N′-tetraacetic acid), BAPTA (1,2-bis(o-aminophenoxy) ethane-N,N,N′,N′-tetraacetic acid), DTPA (diethylenetriaminepenta-acetic acid), and TTHA (triethylenetetraminehexaacetate).

Particularly preferred chelating agents for use herein include compounds of general formula (I):

(HO₂CCH₂)₂N—R—N(CH₂CO₂H)₂ (I),

which are aminopolycarboxylic acid compounds having at least four carboxylic acid functional groups. The R group is not particularly limited and may comprise no carboxylic acid functional group or one or two carboxylic acid functional groups. Preferably, the R group comprises no or one carboxylic acid functional group, most preferably no carboxylic acid functional group.

Examples of compounds of general formula (I) include, for example, EDTA, EGTA, BAPTA, DTPA, and TTHA. Particularly preferred for use herein are EDTA, EGTA, and DTPA, more preferred are EDTA and DTPA, and most preferred is EDTA. Mixtures of any of the above-mentioned chelating agents may also be used within the present invention, for example as solutions, solids or coupled to matrices.

In step (a), the HSA is preferably contacted with an amount of the chelating agent such that the chelating agent is present in the liquid formulation at a concentration of 0.1 mM-1000 mM or 0.1 mM-500 mM, more preferably 0.5 mM-500 mM or 1 mM-500 mM, and most preferably 10 mM-100 mM.

In accordance with the present invention, the method for preparing the liquid formulation further comprises the following step:
(b) removing the chelating agent from the mixture.

In step (b), the chelating agent may be removed by any appropriate technique such as dialysis (including conventional dialysis using a dialysis bag, counterflow dialysis etc.), reverse osmosis, filtration, crossflow filtration, ultrafiltration, and chromatographic methods (e.g., ion exchange chromatography or gel filtration chromatography).

Preferably, the chelating agent is removed by dialysis. The dialysis is typically carried out for 0.5-48 hours, in particular 1-24 hours or 1-12 hours, at a temperature of 0° C. to 30° C., particularly at a temperature of 2° C. to 30° C. or 4° C. to 25° C., including room temperature. Further, the dialysis is typically carried out against a volume of dialyzing buffer of 10 to 1000 times the volume of the incubated HSA/chelating agent mixture, and the dialysis buffer is usually changed at least once. The dialysis membrane used may have a molecular weight cut-off of, e.g., 10 kDa.

Generally, the dialysis conditions (e.g., time, temperature, volume of buffer, number of buffer changes) are selected such that the residual amount of chelating agent (e.g., EDTA) is contained in the final product in a concentration of 100 μM or less, preferably at a concentration of 10 μM or less, more preferably at a concentration of 1 μM or less.

In accordance with a preferred embodiment of the present invention, a HSA starting material is first pre-treated by contacting same with a chelating agent, followed by removing the chelating agent. The thus obtained pre-treated HSA is then used to prepare the liquid botulinum toxin formulation of the present invention. As used herein, the term “human serum albumin starting material” is intended to refer to donor HSA material (HSA derived from human blood or, more precisely, from human plasma) or recombinant HSA material, as commercially or conventionally obtainable, i.e., without having been subjected to a pre-treatment as described herein. A mixture of the donor HSA material and the recombinant HSA material is also encompassed by the term “human serum albumin starting material”, as used herein.

More specifically, according to this preferred embodiment, the liquid formulation comprising (i) botulinum toxin and (ii) human serum albumin is prepared by a method comprising the following steps:
(a) contacting a human serum albumin starting material with a chelating agent to obtain a mixture of the human serum albumin starting material and the chelating agent,
(b) removing the chelating agent from the mixture to obtain a pre-treated human serum albumin material, and
(c) mixing botulinum toxin with the pre-treated human serum albumin material.

In accordance with another preferred embodiment of the present invention, a liquid pre-formulation is contacted with a chelating agent, followed by removing the chelating agent to obtain the liquid formulation. The term “liquid pre-formulation”, as used herein, refers to a liquid formulation which contains at least components (i) and (ii) (i.e., botulinum toxin and HSA), and preferably contains all the components and substances that are included in the final liquid composition, in particular components (iii) and (iv). In case of the latter, the treatment and removal of the chelating agent results in the final liquid formulation.

More specifically, according to this preferred embodiment, the liquid formulation comprising (i) botulinum toxin and (ii) human serum albumin is prepared by a method comprising the following steps:
(a) contacting a liquid pre-formulation comprising botulinum toxin and human serum albumin with the chelating agent to obtain a mixture of the liquid pre-formulation and the chelating agent, and
(b) removing the chelating agent from the mixture to obtain the liquid formulation.

In accordance with the present invention, the liquid formulation (according to any aspect described herein, i.e., according to the first, second and third aspect) may optionally further comprise:
(iii) a tonicity agent.

The term “tonicity agent”, as used herein, refers to an agent that is added to injectable formulations to render the formulations similar in osmotic characteristics to physiologic fluids. The tonicity agent may also be referred to as “osmotic regulator”. The tonicity agent is not particularly limited and may, for example, be selected from the group consisting of sugars, salts, polymers, and mixtures thereof.

Exemplary tonicity agents include sucrose, glucose, sodium carbonate, amino acids, polyethyleneglycol (PEG), dextran, cyclodextrin, and colloids (e.g., colloidal polysaccharides). Typically, the concentration of the tonicity agent is in the range of 0-2.0% w/v, in particular 0.01-2.0% w/v or 0.1-1.5% w/v, more particularly 0.6-1.2% w/V.

Preferably, the tonicity agent is sodium chloride (NaCl). The sodium chloride may be present in the liquid formulation of the present invention in an amount of 0.01-2.0% w/v, preferably 0.1-1.5% w/v, more preferably 0.5-1.2% w/v or 0.8-1.0% w/v, and most preferably 0.9% w/v.

In accordance with the present invention, the liquid formulation (according to any aspect described herein, i.e., according to the first, second and third aspect) may optionally further comprise:
(iv) a buffering agent.

The term “buffering agent”, as used herein, means an agent which maintains the pH of the liquid formulation in an acceptable range, i.e., an agent capable of controlling the pH of the formulation. Suitable buffers are those that are not chemically reactive with other ingredients and are present in amounts sufficient to provide the desired degree of pH buffering. Such buffers include, for example, amino acids, acetate, malic acid, ascorbate, citrate, tartrate, fumarate, succinate, phosphate, bicarbonate, TRIS, Bis-TRIS, ACES, MES, BES, MOPS, HEPES, TES, PIPES, tricine, and imidazole.

Preferably the buffering agent is phosphate (i.e., a phosphate buffer), an amino acid, or a mixture thereof. The term “phosphate”, as used herein generally means unprotonated and protonated forms and any salts thereof. The amino acid may be selected from aspartate, glycine, glutamate, histidine, proline, taurine, methionine, serine, tyrosine, tryptophan, and mixtures thereof, and is preferably selected from histidine, proline, taurine, methionine, serine, tyrosine, and mixtures thereof. Most preferably, the amino acid is histidine. The most preferred buffering agent for use herein is histidine, phosphate or a mixture thereof.

The concentration of the buffering agent in the liquid formulation of the present invention is preferably 1-100 mM, preferably 2-50 mM, more preferably 5-20 mM. If the buffering agent is an amino acid (e.g., histidine), it may be present in the liquid formulation at a concentration of 1-100 mM, preferably 2-50 mM, more preferably 5-20 mM, and most preferably 10 mM. If the buffering agent is phosphate, it may be present in the liquid formulation at a concentration of 1-100 mM, preferably 2-50 mM, more preferably 5-20 mM, and most preferably 10 mM.

Moreover, the liquid formulation of the present invention may further comprise one or more additional pharmaceutically acceptable excipients, unless otherwise stated or intended. For example, the liquid formulation may contain one or more of glycerol, sucrose, lactose, mannitol, dextran, hyaluronic acid, polyvinylpyrrolidone, lactic acid, citric acid, amino acid(s), benzyl alcohol, lidocaine, gelatine, hydroxyethyl starch (HES), polyethylene oxide, and polysorbate (e.g., polysorbate 20, polysorbate 80). Other suitable pharmaceutically acceptable excipients comprise those well known in the art, see, e.g., Remington's Pharmaceutical Sciences, Mack Publishing Company, Easton, Pennsylvania.

On the other hand, it is also contemplated herein that the liquid formulation of the present invention specifically lacks certain components (i.e., compounds, materials or substances), for example chelating agents, detergents, polysaccharides, amino acids, stabilizing peptides, and the like, including any combination thereof. As used herein, the term “detergent”, is synonymously used with “surfactant” and is intended to include non-ionic and ionic detergents. The term “stabilizing peptide”, as used herein, generally means a peptide consisting of 5 to 50 amino acids, such as a peptide of 10 to 40 amino acids or 15 to 30 amino acids. Hence, the term “stabilizing peptide” excludes HSA.

In one embodiment, the liquid formulation of the present invention does not contain detergents, particularly does not contain polysorbate, more particularly does not contain polysorbate 20 and/or polysorbate 80. In another embodiment, the liquid formulation of the present invention does not contain alginate. In another embodiment, the liquid formulation of the present invention does not contain succinate. In another embodiment, the liquid formulation of the present invention does not contain one or more (e.g., 2, 3, 4 or 5) amino acids selected from the group consisting of: arginine, glutamic acid, methionine, tryptophane, and serine. In another embodiment, the liquid formulation of the present invention does not contain a saccharide, such as a mono-, oligo- or polysaccharide or a mixture thereof. In particular, the liquid formulation of the present invention may not contain one or more (e.g., 2, 3 or 4) of sucrose, lactose, maltose, and trehalose. In another embodiment, the liquid formulation of the present invention does not contain a chelating agent, in particular those described herein in connection with the present invention. It is also contemplated within the present invention that the liquid formulation lacks more than one, or all of the compounds mentioned above.

In one embodiment, the liquid formulation of the present invention lacks (i) detergents and mono-, oligo- and polysaccharides, (ii) detergents and any amino acid, or detergents and all amino acids except histidine, (iii) detergents and stabilizing peptides, (iv) mono-, oligo- and polysaccharides and any amino acid, or mono-, oligo- and polysaccharides and all amino acid except histidine, (v) mono-, oligo- and polysaccharides and stabilizing peptides, (vi) any amino acids and stabilizing peptides, or all amino acids except histidine and stabilizing peptides, (vii) detergents, mono-, oligo- and polysaccharides and any amino acid, or detergents, mono-, oligo- and polysaccharides and all amino acids except histidine, (viii) detergents, mono-, oligo- and polysaccharides, and stabilizing peptides, (ix) detergents, any amino acid, and stabilizing peptides, or detergents, all amino acid except histidine, and stabilizing peptides, (x) mono-, oligo- and polysaccharides, any amino acid, and stabilizing peptides, or mono-, oligo- and polysaccharides, all amino acids except histidine, and stabilizing peptides, (xi) detergents, mono-, oligo- and polysaccharides, any amino acid, and stabilizing peptides, or detergents, mono-, oligo- and polysaccharides, all amino acids except histidine, and stabilizing peptides.

In another embodiment, the liquid formulation of the present invention does not contain any other amino acid than histidine. In another embodiment, the liquid formulation of the present invention does not contain any mono-, di- and trisaccharides. In another embodiment, the liquid formulation of the present invention does not contain any other stabilizing peptide or protein than HSA. In another embodiment, the liquid formulation of the present invention does not contain phosphate such as in the form of a phosphate buffer.

In yet other embodiments, the liquid formulation of the present invention lacks (i) succinate and a detergent (e.g., polysorbate), (ii) succinate and methionine, (iii) succinate and sucrose, (iv) a detergent (e.g., polysorbate) and methionine, (v) a detergent (e.g., polysorbate) and sucrose, (vi) methionine and sucrose, (vii) succinate, a detergent (e.g., polysorbate) and methionine, (xiii) succinate, a detergent (e.g., polysorbate) and sucrose, (ix) succinate, methionine and sucrose, (x) a detergent (e.g., polysorbate), methionine and sucrose, and (xi) succinate, a detergent (e.g., polysorbate), methionine and sucrose, (xii) a detergent (e.g. polysorbate and histidine), (xiii) a detergent (e.g. polysorbate), histidine and sucrose).

Furthermore, any of the liquid formulation if the present invention that lacks one or more components (i.e., compounds, materials or substances) may further lack a chelating agent, in particular the chelating agents described herein.

Further, a preferred liquid formulation of the present invention comprises (i) botulinum toxin, (ii) HSA, and (iii) a tonicity agent. A preferred liquid formulation of the present invention comprises (i) botulinum toxin at a concentration of 10-200 U/ml, (ii) HSA in an amount of 0.01-1.0% w/v, and (iii) a tonicity agent, preferably sodium chloride, in an amount of 0.01-2.0% w/v. A preferred liquid formulation of the present invention comprises (i) botulinum toxin at a concentration of 10-200 U/ml, (ii) HSA in an amount of 0.01-0.5% w/v, and (iii) a tonicity agent, preferably sodium chloride, in an amount of 0.1-1.5% w/v. A preferred liquid formulation of the present invention comprises (i) botulinum toxin at a concentration of 10-200 U/ml, (ii) HSA in an amount of 0.05-0.25% w/v, and (iii) a tonicity agent, preferably sodium chloride, in an amount of 0.6-1.2% w/v. A preferred liquid formulation of the present invention comprises (i) botulinum toxin at a concentration of 10-200 U/ml, (ii) HSA in an amount of 0.01-0.5% w/v, and (iii) sodium chloride as a tonicity agent in an amount of 0.9% w/v. A preferred liquid formulation of the present invention comprises (i) botulinum toxin at a concentration of 10-200 U/ml, (ii) HSA in an amount of 0.05-0.25% w/v, and (iii) sodium chloride as a tonicity agent in an amount of 0.9% w/v.

It is also contemplated herein that the preferred liquid formulations described in the preceding paragraph as well as in the following paragraphs may be characterized by a concentration of Fe³⁺ ions of less than 1 μM, preferably less than 1000 nM or less than 500 nM, more preferably less than 250 nM or less than 100 nM, and most preferably less than 10 nM or less than 1 nM.

A preferred liquid formulation of the present invention comprises (i) botulinum toxin, (ii) HSA, (iii) a tonicity agent, and (iv) a buffering agent. A preferred liquid formulation of the present invention comprises (i) botulinum toxin at a concentration of 10-200 U/ml, (ii) HSA in an amount of 0.01-1.0% w/V, (iii) a tonicity agent, preferably sodium chloride, in an amount of 0.01-2.0% w/v, and (iv) a buffering agent in a concentration of 1-100 mM. A preferred liquid formulation of the present invention comprises (i) botulinum toxin at a concentration of 10-200 U/ml, (ii) HSA in an amount of 0.01-0.5% w/V, (iii) a tonicity agent, preferably sodium chloride, in an amount of 0.01-2.0% w/v, and (iv) a buffering agent in a concentration of 1-100 mM. A preferred liquid formulation of the present invention comprises (i) botulinum toxin at a concentration of 10-200 U/ml, (ii) HSA in an amount of 0.05-0.25% w/V, (iii) a tonicity agent, preferably sodium chloride, in an amount of 0.1-2.0% w/v, and (iv) a buffering agent in a concentration of 1-100 mM. A preferred liquid formulation of the present invention comprises (i) botulinum toxin at a concentration of 10-200 U/ml, (ii) HSA in an amount of 0.05-0.25% w/V, (iii) a tonicity agent, preferably sodium chloride, in an amount of 0.6-1.3% w/v, and (iv) a buffering agent in a concentration of 2-50 mM. A preferred liquid formulation of the present invention comprises (i) botulinum toxin at a concentration of 10-200 U/ml, (ii) HSA in an amount of 0.05-0.25% w/V, (iii) sodium chloride in an amount of 0.9% w/v, and (iv) a buffering agent in a concentration of 5-20 mM. It is also contemplated herein that the preferred liquid formulations described in this paragraph may be characterized by a concentration of Fe³⁺ ions of less than 1 μM, preferably less than 1000 nM or less than 500 nM, more preferably less than 250 nM or less than 100 nM, and most preferably less than 10 nM or less than 1 nM.

A preferred liquid formulation of the present invention comprises (i) botulinum toxin, (ii) HSA, (iii) sodium chloride as a tonicity agent, and (iv) a buffering agent selected from histidine, phosphate and a mixture thereof as a buffering agent, wherein the buffering agent is preferably histidine.

A particularly preferred liquid formulation of the present invention comprises (i) botulinum toxin at a concentration of 10-200 U/ml, (ii) HSA in an amount of 0.01-1.0% w/V, (iii) sodium chloride in an amount of 0.01-2.0% w/v, preferably 0.9% w/v, and (iv) a buffering agent selected from histidine, phosphate and a mixture thereof at a concentration of 1-100 mM, wherein the buffering agent is preferably histidine at a concentration of 1-100 mM.

Another particularly preferred liquid formulation of the present invention comprises (i) botulinum toxin at a concentration of 10-200 U/ml, (ii) HSA in an amount of 0.01-0.5% w/V, (iii) sodium chloride in an amount of 0.9% w/v, and (iv) a buffering agent selected from histidine, phosphate and a mixture thereof at a concentration of 1-100 mM, wherein the buffering agent is preferably histidine at a concentration of 1-100 mM.

Yet another particularly preferred liquid formulation of the present invention comprises (i) botulinum toxin at a concentration of 10-200 U/ml, (ii) HSA in an amount of 0.05-0.25% w/V, (iii) sodium chloride in an amount of 0.9% w/v, and (iv) a buffering agent selected from histidine, phosphate and a mixture thereof at a concentration of 1-100 mM, preferably at a concentration of 2 mM to 50 mM or, more preferably 5 mM to 20 mM, wherein the buffering agent is preferably histidine at a concentration of 1-100 mM, preferably at a concentration of 2 mM to 50 mM, more preferably 5 mM to 20 mM,

Furthermore, the preferred and particularly preferred liquid formulations describe above have a pH that is preferably in the range of 6.0-7.5. Moreover, the botulinum toxin is preferably of serotype A and, more preferably, is the neurotoxic component of serotype A.

A preferred exemplary liquid formulation of the present invention is the following:

### Formulation 1:

50 U/ml BoNT/A
0.9% NaCl (9 mg/mL)
0.085% HSA (EDTA-dialyzed) (0.85 mg/mL)
0.155% histidine (1.55 mg/ml; ˜ 10 mM)
pH 6.0

The HSA (EDTA-dialyzed) of exemplary Formulation 1 is prepared by an exemplary process including the following steps:
addition of 37.2 mg Na₂-EDTA per ml of 20% HSA solution with stirring and adjustment to pH 8.0 with NaOH,
incubation for 6 hours at room temperature with stirring,
dialysis against 100-fold volume of EDTA buffer (100 mM Na₂-EDTA; 10 mM histidine; 0.9% NaCl pH 7.0) for 16 h at room temperature with gentle stirring on magnetic stirrer (MWCO of dialysis membrane 10 kDa)
dialysis against 100-fold volume of 10 mM histidine, 0.9% NaCl pH 6.0 for 24 h at room temperature, followed by replacing dialysis buffer with fresh buffer and dialyzing for another 24 h, and repeating two times more.

In exemplary Formulation 1, EDTA can optionally be replaced by other chelators such as EGTA, BAPTA or DTPA. As an alternative to adding solid EDTA to the HSA solution, a concentrated EDTA solution can be used (e.g., 200 mM EDTA, pH 8.0). The first dialysis step (against EDTA buffer) can also be omitted. Other methods, such as crossflow dialysis or ultrafiltration, can also be used instead of dialysis.

The present invention also relates to a liquid formulation comprising a botulinum toxin, wherein the toxin activity is not reduced by more than 20%, relative to the initial toxin activity, upon storage of the liquid formulation for 4 weeks at an elevated temperature of 40° C. Furthermore, the present invention relates to a liquid formulation comprising a botulinum toxin, wherein the toxin activity is not reduced by more than 20%, relative to the initial toxin activity, upon exposure of the liquid formulation for 7 hours to a light source at 250 W/m². Preferably, for the photostability testing, i.e., for the exposure for 7 hours at 250 W/m², a SUNTEST CPS+ instrument (ATLAS Material Testing Technology LLC) can be used, equipped with a filter set to provide a spectral distribution in the wavelength range of 320-800 nm corresponding to the ID65 (indoor indirect daylight standard) per ISO 10977 with a window glass filter according to ICH Q1B.

Moreover, the present invention further relates to a method for preparing the liquid botulinum toxin formulation described herein, comprising adding the component described herein, in particular components (i) to (ii) and optionally one or more of components (iii) and (vi), preferably components (i), (ii), (iii) and (iv). The preparation of the liquid formulation of the present invention is not particularly limited and the respective formulation techniques are known to those skilled in the art. As described above, the liquid formulation of botulinum toxin is generally an aqueous solution, preferably a saline solution, more preferably a physiological saline solution, and most preferably a buffered (e.g., phosphate or histidine buffered) physiological saline solution.

Preferably, the salts are dissolved first, then HSA is added, the pH is adjusted if necessary, and finally the botulinum toxin is added. This order is not a mandatory requirement but is believed to safely preserve maximum specific activity of the BoNT. Preferably, the method for preparing the liquid botulinum toxin formulation does not comprise the reconstitution of a lyophilized botulinum toxin preparation in powder form.

Further, the present invention relates to a method for stabilizing a liquid botulinum toxin formulation, the method comprising combining botulinum toxin with a human serum albumin (HSA), wherein the HSA is prepared by a method comprising the following steps:
(a) contacting a composition comprising human serum albumin with a chelating agent to obtain a mixture of human serum albumin and the chelating agent, and
(b) removing the chelating agent from the mixture.

Still further, the present invention relates to a use of a human serum albumin (HSA) for increasing the light and/or the temperature stability of a liquid composition comprising a botulinum toxin, wherein the HSA is prepared by a method comprising the following steps:
(a) contacting a composition comprising human serum albumin with a chelating agent to obtain a mixture of human serum albumin and the chelating agent, and
(b) removing the chelating agent from the mixture.

Yet further, the present invention relates to a human serum albumin (HSA) which is obtainable by
(a) contacting a composition comprising human serum albumin with a chelating agent to obtain a mixture of human serum albumin and the chelating agent, and
(b) removing the chelating agent from the mixture.

In a fourth aspect, the present invention relates to a method for preparing a liquid formulation of the present invention, wherein the method comprises the following steps:
purifying a human serum albumin starting material to obtain a purified human serum albumin material,
mixing the obtained purified human serum albumin with botulinum toxin and
optionally further components to obtain the liquid formulation, or
purifying a liquid composition comprising botulinum toxin and human serum albumin to obtain a purified liquid composition,
mixing the obtained purified liquid composition with further components to obtain the liquid formulation.

The human serum albumin starting material is defined as herein above. Preferably, it is in the form of an aqueous solution containing at least 5% w/v HSA, preferably 10% w/v to 35% w/v HSA, more preferably 15% w/v to 30% w/v, and most preferably 20% w/v to 25% w/v HSA. The human serum albumin starting material may contain tryptophan and N-acetyl-tryptophan, in particular N-acetyl-tryptophan, in an amount of more than 5 mM, in an amount of 10 mM or more, in an amount of 20 mM or more, or in an amount of 30 mM or more. Furthermore, the pH of the mixture obtained in step (a) may be adjusted to a pH in the range of 6.0 to 9.0, preferably 6.5 to 8.5, more preferably 7.0 to 8.5, and most preferably 7.0 to 8.0.

Preferably, in the first alternative of the method of the present invention, the purifying step results in a purified human serum albumin material which contains no or no more than 5 mM, preferably no more than 1 mM, more preferably no more than 0.1 mM, and most preferably no more than 0.001 mM tryptophan and/or N-acetyl-tryptophan. Upon preparation of the liquid formulation, the purified human serum albumin material is diluted to result in a concentration of tryptophan and N-acetyl-tryptophan falling within the ranges described herein, e.g., of less than 50 μM.

The purifying step may be carried out by subjecting the human serum albumin starting material or the liquid composition comprising botulinum toxin and human serum albumin to dialysis, diafiltration (crossflow filtration), ultrafiltration, chromatographic purification techniques such as ion exchange chromatography (IEX), affinity chromatography and hydrophobic interaction chromatography (HIC), field flow fractionation or precipitation (e.g., salt precipitation, ethanol precipitation).

Preferably, the purification is carried out by dialysis. The dialysis is typically carried out for 0.5 to 48 hours, in particular 1 to 24 hours or 1 to 12 hours, at a temperature of 0° C. to 30° C., particularly at a temperature of 2° C. to 30° C. or 4° C. to 25° C., including room temperature. Further, the dialysis is typically carried out against a volume of dialyzing buffer of 10 to 1000 times the volume of the incubated HSA solution, and the dialysis buffer is usually changed at least once. The dialysis membrane used may have a molecular weight cut-off of, e.g., 10 kDa.

For example, the purified serum albumin may be obtained by the following process:
dialysis of a commercially available HSA product against 100-fold volume of dialysis buffer (10 mM histidine, 0.9% NaCl, pH 6.0) for 24 h at room temperature with gentle stirring on magnetic stirrer (MWCO of dialysis membrane 10 kDa),
changing the buffer by fresh dialysis buffer and dialysis for 24 h at room temperature, and repeating two times more.

The first alternative of the above method, i.e., the purification of a human serum albumin starting material and mixing the obtained purified human serum albumin with botulinum toxin and optionally further components, is preferred within the present invention. The mixing step to obtain the liquid formulation of the present invention is not particularly limited and includes mixing components (i) to (ii) and optionally one or more of components (iii) and (vi). Preferably, the salts are dissolved first, then HSA is added, the pH is adjusted if necessary, and finally the botulinum toxin is added. This order is not a mandatory requirement but is believed to safely preserve maximum specific activity of the BONT. Preferably, the method for preparing the liquid botulinum toxin formulation does not comprise the reconstitution of a lyophilized botulinum toxin preparation in powder form.

As mentioned above with respect to the first aspect of the present invention, it is also contemplated herein that the liquid formulation of the present invention has a low Fe³⁺ ion concentration since Fe³⁺ ion also exert a destabilising effect on the light stability. Therefore, in accordance with the present invention, to remove Fe³⁺ ions, the purifying step of the method according to the present invention is conducted as follows or additionally contains the following steps:
contacting a human serum albumin starting material with a chelating agent to obtain a mixture of the human serum albumin starting material and the chelating agent, and
removing the chelating agent from the mixture to obtain a chelating agent-treated purified human serum albumin material, or
contacting a liquid composition comprising botulinum toxin and human serum albumin with the chelating agent to obtain a mixture of the liquid composition and the chelating agent, and
removing the chelating agent from the mixture to obtain a chelating agent-treated purified liquid formulation.

In the contacting step, the human serum albumin starting material and the liquid composition comprising botulinum toxin and human serum albumin, respectively, are preferably contacted with an amount of the chelating agent such that the chelating agent is present in the mixture at a concentration of 0.1 mM to 1000 mM or 0.1 mM to 500 mM, more preferably 0.5 mM to 500 mM or 1 mM to 500 mM, and most preferably 10 mM to 100 mM.

The removal of the chelating agent may be carried out by dialysis, crossflow filtration or ultrafiltration.

As used herein, the term “contacting” is intended to be broadly interpreted as bringing two or more components together. This can be achieved by a variety of different methods, such as dissolving, mixing, suspending, blending, slurrying, stirring, flowing by, adsorbing, binding, extracting etc. Typically, the chelating agent is added to a composition comprising HSA (e.g., a solution of HSA), followed by mixing to obtain a homogeneous mixture, and optionally further processing steps (e.g., dialysis against a chelating agent containing buffer).

Accordingly, it is also contemplated herein that “contacting” can be carried out by loading the HSA material, e.g., in the form of an aqueous solution, onto an immobilized metal affinity resin (IMAC). The term “immobilized metal affinity resin” (IMAC), as used herein, includes, but is not limited to, resins containing an immobilized functional moiety (e.g., iminodiacetic acid) capable of binding and coordinating multivalent cations, for example Chelating-Sepharose, Fractogel-EMD-Chelate, POROS 20MC, Matrex Cellufine Chelate, TALON, and Chelex 100 resin. Such immobilized metal affinity resins are commonly used in the form of chromatographic metal affinity columns as known to those skilled in the art. Furthermore, as used herein, the term “contacting” also includes batch mode binding using a suitable immobilized metal affinity resin.

As regards the contacting step, it is further pointed out that the terms “human serum albumin starting material” and “chelating agent”, as used in the definition of the contacting step, are not intended to imply any restrictions as to their physical form or to exclude the presence of other substances or compounds that are mixed with, or included in, the HSA and the chelating agent. This means that the “human serum albumin starting material” that is contacted with the chelating agent may be present in any form, such as in the form of a solid or a liquid (e.g., an aqueous composition or aqueous solution). Likewise, the “chelating agent” that is contacted with the human serum albumin starting material may be present in any form, such as in the form of a solid or a liquid (e.g., an aqueous composition or aqueous solution).

Preferably, the mixture obtained in the contacting step is an aqueous mixture. This aqueous mixture can be prepared by different ways. For example, the human serum albumin may be in the form of an aqueous composition, e.g., an aqueous solution, which is mixed with the chelating agent that may be present in solid form or in liquid form, e.g., in the form of an aqueous solution of the chelating agent. Furthermore, the human serum albumin may be in the form of a solid, e.g., a lyophilized material, which is mixed with a chelating agent and an aqueous solution or with an aqueous solution of the chelating agent. Preferably, the human serum albumin is in the form of an aqueous composition, more preferably an aqueous solution, and the chelating agent is a solid or an aqueous composition (e.g., aqueous solution).

In particular, the HSA that is contacted in the contacting step with a chelating agent may be in the form of an aqueous solution containing at least 5% w/v HSA, preferably 10% w/v to 35% w/v HSA, more preferably 15% w/v to 30% w/v HSA, and most preferably 20% w/v to 25% w/v HSA. Furthermore, the pH of the mixture obtained in step (a) may be adjusted to a pH in the range of 6.0 to 9.0, preferably 6.5 to 8.5, more preferably 7.0 to 8.5, and most preferably 7.0 to 8.0.

Moreover, the contacting step of the method for preparing the liquid formulation according to the present invention may comprise several sub-steps. For example, in one embodiment, the contacting step comprises, or consists (only) of, the steps of mixing a chelating agent (e.g., EDTA) and human serum albumin, incubating said mixture for a determined time, and optionally subjecting said mixture to dialysis against a buffer containing a chelating agent, the chelating agent being preferably the same as that used in the incubating sub-step. In another embodiment, said mixture is not incubated but (directly) subjected to dialysis against a buffer containing a chelating agent, the chelating agent being preferably the same as that used in the step of mixing a chelating agent (e.g., EDTA) and human serum albumin. In another embodiment, the chelating agent and human serum albumin are not mixed prior to dialysis. This is, contacting step (a) may comprise, or consist (only) of, the step of subjecting said mixture to dialysis against a buffer containing a chelating agent.

Preferably, the contacting step comprises, or consists (only) of, a step of adding a chelating agent to a composition comprising HSA (e.g., a solution of HSA) or of mixing a chelating agent with a composition comprising HSA (e.g. a solution of HSA). The resulting mixture is then incubated for some time, e.g., allowed to stand for a given time without stirring or stirred for a given time.

The time of incubation is not confined to a specific range but is typically at least 0.5 hours, particularly at least 1 hour, and more particularly at least 2 hours. The upper limit of the time of incubation is not critical and may be, e.g., 1 hour, 2 hours, 5 hours or 10 hours. Hence, the incubation time may be, for examples, 0.5 to 5 hours or 1 hour to 10 hours. Likewise, the incubation temperature is not particularly limited and may, for example, be within the range of 0° C. to 60° C. Preferably, the temperature is from 0° C. to 30° C. This means that room temperature (20° C. or 25° C.) is a suitable temperature within the present invention. As known to the skilled person, the temperature influences the reaction time. Generally, the incubation conditions (e.g., time and temperature) are selected such that the residual amount of chelating agent (e.g., EDTA) that is contained in the final product is 100 μM or less, preferably 10 μM or less, more preferably 1 μM or less.

Optionally, the incubation step is followed by further processing such as dialysis of the incubated mixture against a buffer containing a chelating agent, typically the same chelating agent as that used in the incubation step. The chelating agent used in this optional dialyzing step is preferably contained in the dialysis buffer at a concentration of 0.1 mM to 1000 mM, more preferably 1 mM to 200 mM, and most preferably 10 mM to 100 mM. It is also preferred that the buffer used in the dialyzing step (i) has a pH of 7.5 to 8.5, (ii) further comprises a buffering agent, preferably according to the final composition, or (iii) further comprises a tonicity agent, preferably 0.9% sodium chloride w/v, or (i) and (ii), or (i) and (iii), or (ii) and (iii), or (i) and (ii) and (iii).

Suitable chelating agents for use herein are as defined herein above. Mixtures of any of the above-mentioned chelating agents may also be used within the present invention, for example as solutions, solids or coupled to matrices.

In accordance with the present invention, the chelating agent may be removed by any appropriate technique such as dialysis (including conventional dialysis using a dialysis bag, counterflow dialysis etc.), reverse osmosis, filtration, crossflow filtration, ultrafiltration, and chromatographic methods (e.g., ion exchange chromatography or gel filtration chromatography).

Preferably, the chelating agent is removed by dialysis. The dialysis is typically carried out for 0.5 hours to 48 hours, in particular 1 hour to 24 hours or 1 hour to 12 hours, at a temperature of 0° C. to 30° C., particularly at a temperature of 2° C. to 30° C. or 4° C. to 25° C., including room temperature. Further, the dialysis is typically carried out against a volume of dialyzing buffer of 10 to 1000 times the volume of the incubated HSA/chelating agent mixture, and the dialysis buffer is usually changed at least once. The dialysis membrane used may have a molecular weight cut-off of, e.g., 10 kDa.

Generally, the dialysis conditions (e.g., time, temperature, volume of buffer, number of buffer changes) are selected such that the residual amount of chelating agent (e.g., EDTA) is contained in the final product in a concentration of 100 μM or less, preferably at a concentration of 10 μM or less, more preferably at a concentration of 1 μM or less.

In a fifth aspect, the present invention relates to a liquid formulation of the present invention for use in therapy.

In particular, the liquid formulation of the present invention may be used in the treatment of neuromuscular diseases, pain, sialorrhea, hyperhidrosis, urological disorders, and neurological disorders.

Exemplary neuromuscular diseases include dystonia, cervical dystonia, spasm, post-stroke spasticity, blepharospasm, tremor, hyperkinetic movement disorders, and cerebral palsy. The urological disorders include, among others, conditions characterized by detrusor overactivity, overactive bladder, neurogenic bladder and interstitial cystitis, treatment of vulvodynia and chronic pelvic pain, benign prostate hyperplasia (BPH) and detrusor sphincter dyssynergia (DSD). Exemplary neurological disorders include chronic migraines, trigeminal pain, peripheral neuropathic pain, diabetic neuropathic pain and depression.

In a sixth aspect, the present invention relates to a cosmetic use of the liquid formulation of the present invention for the treatment of a cosmetic condition.

This aspect of the present invention relates to a purely aesthetic use of the liquid formulation of the present invention. Preferred cosmetic conditions to be treated include skin conditions, in particular the treatment of wrinkles of the skin, in particular of the face.

The term “wrinkles”, as used herein, is to be broadly construed to not only include wrinkles, but also lines, rhytids, creases, furrows, and folds. The words “lines”, “wrinkles”, “rhytids”, “creases”, and “folds” share similar definition and are therefore often used interchangeably. Within the present invention, “lines” are generally interchangeable with “wrinkles” but may preferably refer to a cutaneous depression that is less deep than a “wrinkle”. A “fold” is interchangeable with wrinkles and lines and is preferably a linear depression. A “crease” is interchangeable with wrinkles, lines and folds. It preferably refers to a mild form of wrinkles and may describe the specific wrinkle in certain locations. A “rhytid”, as used herein, has essentially the same meaning of wrinkle. However, a “rhytid” preferably refers to a skin structure that is formed by irregular aggregation of lines. A “furrow” is a deep fold or deep line in the skin.

Preferably, the wrinkles treated in accordance with the present invention are facial wrinkles including include horizontal forehead lines, glabellar frown lines, periorbital lines, Crow's feet, bunny lines (i.e., downward radiating lines on the sides of nose), nasolabial folds, upper radial lip lines, lower radial lip lines, corner of the mouth lines, marionette lines, perioral lip lines, oral commissures, labiomental crease and cobblestone chin.

In order to treat the above-mentioned facial wrinkles, botulinum toxin is usually administered by intramuscular injection to the following muscles: frontalis muscle (horizontal forehead lines), procerus and corrugator muscles (glabellar frown lines), lateral orbicularis oculi muscle (Crow's feet/periorbital lines), nasalis, procerus for transverse nasal muscles (bunny lines), levator labii superioris alaeque nasi (nasolabial folds), orbicularis oris (upper and lower radial lip lines), depressor anguli oris (corner of the mouth lines, marionette lines, oral commissures, labiomental crease), and mentalis muscles (perioral lip lines, cobblestone chin).

A further preferred cosmetic use of the liquid formulation of the present invention relates to the use for cosmetic applications comprising rejuvenation and/or improvement of the skin quality of the face and/or body.

In a seventh aspect, the present invention relates to a method of treating a disease or condition, comprising administering an effective amount of the liquid formulation of the present invention to a person in need thereof.

The disease or condition may be any one of the diseases and conditions mentioned hereinabove, irrespective of whether it is a therapeutic or cosmetic indication. Thus, in one embodiment, the present invention relates to a (non-therapeutic) method of treating a cosmetic (aesthetic) condition, preferably a skin condition, comprising administering an effective amount of the liquid formulation of the present invention to a person in need thereof. In another embodiment, the present invention relates to a (non-therapeutic) method of treating a cosmetic (aesthetic) condition, preferably a skin condition, comprising injecting an effective amount of the liquid formulation of the present invention into a person in need thereof.

A further preferred method of the present invention relates to a method of rejuvenation and/or improvement of the skin quality of the face and/or body, comprising administering an effective amount of the liquid formulation of the present invention to a person in need thereof.

The person to be treated is not particularly limited other than by having a disease or condition that can be treated in accordance with the present invention. Those skilled in the art will be able to determine appropriate administration regimens for the treatment of a given therapeutic or cosmetic indication. In particular, the injection may be intradermal, subdermal (subcutaneous), or intramuscular, depending on the disease or condition to be treated.

Apart from the above, the present invention further relates to a method for stabilizing a liquid botulinum toxin formulation, the method comprising combining botulinum toxin with a human serum albumin (HSA), wherein the HSA is prepared by purifying a human serum albumin starting material to obtain a purified human serum albumin material as described in relation to the fourth aspect of the present invention.

Further, the present invention also relates to a use of a human serum albumin (HSA) for providing a light and temperature stable liquid composition comprising botulinum toxin, wherein the HSA is prepared by purifying a human serum albumin starting material to obtain a purified human serum albumin material as described in relation to the fourth aspect of the present invention.

### EXAMPLES

### I. Examples 1 to 3

The following examples illustrate the liquid botulinum toxin formulation according to the present invention and its preparation method. Percentages are weight by volume (w/v) unless otherwise indicated.

The biological activity of botulinum toxin was determined using a cell-based potency assay (CBA) as described in WO 2014/207109. In brief, neuronal cells were incubated with the neurotoxin containing sample and a reference standard of known potency.

After the incubation period, the cells were lyzed and the amount of cleaved SNAP25 protein was determined by an immunoassay. The biological activity of the sample was then calculated by comparing the cleavage rate of the cells treated with the sample with those treated with the reference standard.

### Example 1

### Light and Storage Stability of Liquid Botulinum Toxin Formulations Containing HSA Pre-Treated by Conventional Dialysis (not According to the Present Invention)

In initial experiments, it was found that the biological activity of liquid formulations of the 150 kDa botulinum neurotoxin type A free of complexing proteins (also referred to herein as “150 kDa BoNT/A”) strongly decreased after exposure to light (daylight or room light) and that the light sensitivity increased with increasing human serum albumin (HSA) concentrations. In further experiments, the present inventors unexpectedly found that the light stability and storage stability at 40° C. of liquid botulinum toxin formulations could be significantly increased by adding complexing agents such as EDTA, even if the EDTA was present in the form of its magnesium, calcium or zinc complexes (results not shown).

In view of these findings, it was assumed that an unknown component in HSA caused the photosensitivity of botulinum toxin and that this component can somehow be inactivated or masked by a chelating agent such as EDTA. However, in further experiments carried out by the present inventors, it was surprisingly found that EDTA resulted in increased injection pain. Therefore, in order to dispense with chelating agents such as EDTA, the present inventors have tried to remove the unknown component in HSA that causes light instability by dialysis.

For this purpose, a concentrated stock solution of HSA (e.g., 20%) was extensively dialyzed at room temperature against a buffer of 10 mM histidine, 0.9% NaCl, pH 6.0, for 4×12 hours (three buffer changes, buffer volume about 100 times the sample volume) to obtain a “dialyzed HSA”. The dialyzed HSA and the non-dialyzed HSA were then used to prepare the following liquid formulations of botulinum toxin (150 kDa BoNT/A without complexing proteins):
Formulation 1:65 U/mL botulinum toxin, 0.1% HSA (non-dialyzed), 10 mM histidine, 0.9% NaCl, pH 6.0
Formulation 2:65 U/mL botulinum toxin, 0.1% HSA (dialyzed), 10 mM histidine, 0.9% NaCl, pH 6.0

The light stability of these two formulations was determined by measuring the relative botulinum toxin activity after exposure to light for 7 hours at 250 W/m² relative to a control sample stored in the dark. For the exposure to light for 7 hours at 250 W/m², a SUNTEST CPS+ instrument (ATLAS Material Testing Technology LLC) was used, equipped with a filter set to provide a spectral distribution in the wavelength range of 320-800 nm corresponding to the ID65 (indoor indirect daylight standard) per ISO 10977 with a window glass filter according to ICH Q1B. The results are shown in Table 1.

TABLE 1
Light stability (7 h, 250 W/m²) of liquid botulinum toxin
formulations containing non-dialyzed HSA or dialyzed HSA

| BoNT/A | | Relative BoNT/A activity |
|---|---|---|
| formulation | Dialysis | (% of control) |
| 1 | no | 46% |
| 2 | yes | 56% |

As is evident from Table 1, only a limited influence on the light stability was observed for Formulation 2 containing dialyzed HSA compared to Formulation 1 containing non-dialyzed HSA.

Furthermore, the storage stability of Formulations 1 and 2 was determined by measuring the relative botulinum toxin activity after storage at 40° C. for 2 and 4 weeks relative to a control sample at TO (measured immediately after preparation of Formulations 1 and 2). The results are shown in Table 2.

TABLE 2
Storage stability at 40° C. of liquid botulinum toxin
formulations containing non-dialyzed HSA or dialyzed HSA

| | | Relative BoNT/A activity (% of control at T0) | |
|---|---|---|---|
| BoNT/A | | after storage for 2 | after storage for 4 |
| formulation | Dialysis | weeks at 40° C. | weeks at 40° C. |
| 1 | no | 66% | 52% |
| 2 | yes | 65% | 51% |

As can be seen, dialysis has no impact on the storage stability of the botulinum toxin.

### Example 2

### Light and Storage Stability of Liquid Botulinum Toxin Formulations Containing HSA Pre-Treated with Complexing Agent and Dialysis

Despite the findings from Example 1 (i.e., EDTA or EDTA complexes required for light stability; dialyzed HSA does not result in a significant improvement of light and storage stability), the present inventors have continued to research for a liquid botulinum toxin formulation that is light and storage stable despite being free of a chelating agent such as EDTA.

First, EDTA was added to a concentrated stock solution of HSA (e.g., 20%) (100 mM=37.2 mg Na₂-EDTA per ml HSA solution at pH 8.0) and incubated for 6 hours at room temperature under stirring. Then, the incubated mixture was dialyzed against a buffer with EDTA (100 mM EDTA, 10 mM histidine, 0.9% NaCl, pH 7.0) for 16 hours at room temperature. The dialysis membrane used was a membrane with a MWCO of 10 kDa and the fill volume was about 0.2 ml per cm² dialysis membrane. The volume of the dialysis buffer was about 100 times the volume of the sample volume.

Thereafter, the EDTA was removed by dialysis for 24 hours at room temperature against a volume of EDTA-free dialysis buffer (10 mM histidine, 0.9% NaCl, pH 6.0) of about 100 times that of the sample volume. The dialysis buffer was then replaced by fresh dialysis buffer and again dialyzed for 24 hours. This was repeated two times to obtain the pretreated HSA (“EDTA-dialyzed HSA”).

This pretreated HSA was then used to prepare liquid Formulation 3 of botulinum toxin (150 kDa BoNT/A without complexing proteins):
Formulation 1: identical to Formulation 1 of Example 2 (65 U/mL botulinum toxin, 0.1% HSA (non-dialyzed), 10 mM histidine, 0.9% NaCl, pH 6.0)
Formulation 3:65 U/mL botulinum toxin, 0.085% HSA (EDTA-dialyzed), 10 mM histidine, 0.9% NaCl, pH 6.0

The light stability of Formulations 1 and 3 was determined by measuring the relative botulinum toxin activity after exposure to light for 7 hours at 250 W/m² relative to a control sample stored in the dark. The results are shown in Table 3.

TABLE 3
Light stability (7 h, 250 W/m²) of liquid botulinum toxin formulations
containing non-dialyzed HSA or EDTA-dialyzed HSA

| BoNT/A | EDTA/ | Relative BoNT/A activity |
|---|---|---|
| formulation | Dialysis | (% of control) |
| 1 | no | 46% |
| 3 | yes | 84% |

As is evident from Table 3, a marked improvement in light stability was observed for Formulation 3 containing EDTA-dialyzed HSA compared to Formulation 1 containing non-pretreated HSA.

Furthermore, the storage stability was determined by measuring the relative botulinum toxin activity after storage at 40° C. for 2 and 4 weeks relative to a control sample at TO (measured immediately after preparation of Formulations 1 and 3). The results are shown in Table 4.

TABLE 4
Storage stability at 40° C. of liquid botulinum toxin
formulations containing non-dialyzed HSA or EDTA-dialyzed HSA

| | | Relative BoNT/A activity (% of control at T0) | |
|---|---|---|---|
| BoNT/A | EDTA- | after storage for 2 | after storage for 4 |
| formulation | dialysis | weeks at 40° C. | weeks at 40° C. |
| 1 | no | 66% | 52% |
| 3 | yes | 94% | 82% |

The results show that the use of EDTA-treated and dialyzed HSA results in a significantly higher biological toxin activity compared to Formulation 1 containing untreated (non-dialyzed) HSA.

Overall, this example shows that the chelating agent (e.g., EDTA) can be unexpectedly omitted from the liquid formulation if the HSA is pre-treated with a chelating agent followed by removal of the chelating agent by, e.g., dialysis. The resulting formulation is not only light and temperature (storage) stable but also exhibits reduced injection pain when injected compared to liquid formulations containing a chelating agent such as EDTA.

### Example 3

### Impact of Iron Ions on the Light Stability of Liquid Botulinum Toxin Formulations

Further experiments to investigate the influence of metal ions on light sensitivity were carried out. To this extent, calcium (1 mM Ca²⁺), cobalt (1 mM Co²⁺), copper (1 mM Cu²⁺), nickel (1 mM Ni²⁺) or iron (1 mM Fe³⁺) was added to a liquid formulation containing 50 U/ml of 150 kDa BoNT/A, 0.085% pre-treated HSA prepared in accordance with Example 2 (i.e., EDTA-treated and dialyzed HSA), 10 mM histidine, 0.9% NaCl, pH 6.0. Said liquid formulation was prepared with high purity water, histidine and NaCl, and without using iron or steel equipment. The resulting formulations were exposed to light (7 hours at 250 W/m²) and compared to dark-stored control formulations.

It was found that the addition of calcium (1 mM Ca²⁺) as well as the addition of cobalt (1 mM Co²⁺) have no effect on light stability (74% and 77% residual toxin activity compared to the dark-stored control formulations), when compared to the formulation without added calcium and cobalt (79% residual toxin activity compared to the dark-stored control formulation). Addition of copper (1 mM Cu²⁺) and nickel (1 mM Ni²⁺) caused a relatively small reduction of light stability. In contrast, addition of iron (1 mM Fe³⁺) caused a drastic reduction of light stability, with only 1% residual toxin activity remaining after exposure to light (7 hours at 250 W/m²) compared to the dark-stored control formulation (results not shown).

In view of these results, the influence of Fe³⁺ ions on the light sensitivity of liquid BONT/A formulations were investigated in more detail in a concentration range between 10 PM and 100 μM. Analogous to the experiments described above, the same formulation comprising pre-treated HSA (50 U/ml botulinum toxin, 0.085% pre-treated HSA, 10 mM histidine, 0.9% NaCl, pH 6.0) was used and the Fe³⁺ concentration was adjusted to the desired final concentration using stock solutions (50 mM Fe(NO₃)₃). The biological activity measured after exposure to light for 7 hours at 250 W/m², expressed as percentage of the biological activity measured for the respective sample stored in the dark is shown in Table 5.

TABLE 5
Light stability (7 h, 250 W/m²) of liquid botulinum toxin formulations
containing pre-treated HSA and varying Fe³⁺ concentrations

| BoNT/A | | Relative BoNT/A activity |
|---|---|---|
| formulation | [Fe³⁺] | (% of dark control) |

| | | | |
|---|---|---|---|
| 1 | 100 | μM | 0.4% |
| 2 | 1 | μM | 9% |
| 3 | 316 | nM | 66% |
| 4 | 100 | nM | 82% |
| 5 | 32 | nM | 77% |
| 5 | 10 | nM | 75% |
| 4 | 100 | pM | 74% |
| 5 | 10 | pM | 78% |
| 6 | 0 | pM | 78% |

As can be seen from Table 4, at a concentration of 316 nM, the Fe³⁺ ions have a significant impact on the BonT/A stability, with a stability decrease of >15%. At a concentration of 1 μM, the light stability is significantly reduced; however, it is still higher than at concentrations above 1 μM. At a concentration of 1 μM and higher, the added Fe³⁺ ions have an extremely negative effect on the stability of BoNT/A in liquid formulations under the influence of light.

In another experiment, it was found that stirring of the liquid botulinum toxin formulation containing the pre-treated HSA (i.e., 50 U/ml botulinum toxin, 0.085% pre-treated HSA, 10 mM histidine, 0.9% NaCl, pH 6.0) for 48 hours in a stainless-steel beaker resulted in a relative BoNT/A activity after exposure to light (7 hours at 250 W/m²) of only about 2% compared to 78% obtained for the same sample stirred for 48 hours in a polypropylene vessel. This is a very surprising finding, likely caused by the leaching of minor amounts of Fe³⁺ ions from the stainless-steel beaker into the liquid botulinum toxin formulation and shows that iron ions are an important factor to consider when preparing liquid botulinum toxin formulations.

### II. Examples 4 to 6

The following examples 4 to 6 further demonstrate the light stability of the liquid botulinum toxin formulation according to the present invention. To this end, several liquid botulinum toxin formulations were prepared (Example 4) and samples of each of said prepared formulations were exposed to light for 7 hours at 250 W/m² or stored in the dark for 7 hours as a control. Then, the botulinum toxin activity of the light exposed samples and the dark stored examples was measured by a cell-based potency assay (CBA). The light stability is expressed as the percentage of the average of the measured botulinum toxin activity of the light exposed samples to the average of the measured botulinum toxin activity of the dark stored samples.

The biological activity of botulinum toxin was determined using a cell-based potency assay (CBA) as described in WO 2014/207109. In brief, neuronal cells were incubated with the neurotoxin containing sample and a reference standard of known potency. After the incubation period, the cells were lyzed and the amount of cleaved SNAP25 protein was determined by an immunoassay. The biological activity of the sample was then calculated by comparing the cleavage rate of the cells treated with the sample with those treated with the reference standard.

For the exposure to light for 7 hours at 250 W/m², a SUNTEST CPS+ instrument (ATLAS Material Testing Technology LLC) was used, equipped with a filter set to provide a spectral distribution in the wavelength range of 320-800 nm corresponding to the ID65 (indoor indirect daylight standard) per ISO 10977 with a window glass filter according to ICH Q1B.

### Example 4

### Preparation of Different Botulinum Toxin Formulations

The composition of the formulations to be analyzed in respect of their light stability are shown in Tables 6 and 7. The botulinum toxin (BoNT) used was NT101.

TABLE 6
Test formulations with untreated/pretreated HSA

Formulation

| | | | | |
|---|---|---|---|---|
| Component | I | II | III | IV |

| BoNT (U/ml) | | | 50 | 50 | 50 | 50 |
|---|---|---|---|---|---|---|
| | HSA | untreated | 1.0 | — | 1.0 | — |
| | (mg/ml) | dialyzed | — | 1.0 | — | — |
| | | EDTA-dial. | — | — | — | 0.85 |

| | | | | |
|---|---|---|---|---|
| NaCl (mg/ml) | 9 | 9 | 9 | 9 |
| Histidine (mg/ml) | 1.55 | 1.55 | 1.55 | 1.55 |
| EDTA (mg/ml) | — | — | 1.30 | — |
| pH | 6.0 | 6.0 | 6.0 | 6.0 |

TABLE 7
Test formulations with tryptophan
(Trp)/N-acetyl-tryptophan (N-AcTrp)

| | Formulation | | | | | | |
|---|---|---|---|---|---|---|---|
| Component | 1 | 2 | 3 | 4 | 5 | 6 | 7 |
| BoNT (U/ml) | 50 | 50 | 50 | 50 | 50 | 50 | 50 |
| HSA EDTA-dial. | 0.85 | 0.85 | 0.85 | 0.85 | 0.85 | 0.85 | 0.85 |
| (mg/ml) | | | | | | | |
| NaCl (mg/ml) | 9 | 9 | 9 | 9 | 9 | 9 | 9 |
| Histidine (mg/ml) | 1.55 | 1.55 | 1.55 | 1.55 | 1.55 | 1.55 | 1.55 |
| Trp (mM) | — | — | — | — | 10 | 0.08 | — |
| N-AcTrp (mM) | 10 | 2 | 0.4 | 0.08 | — | — | — |
| pH | 6.0 | 6.0 | 6.0 | 6.0 | 6.0 | 6.0 | 6.0 |

Preparation of “HSA-dialyzed”. 10 ml of HSA 25% (CSL Behring) were transferred into a dialysis cassette (Slide-A-Lyzer™, 10 kDa MWCO, 12-30 ml, Thermo Scientific/Pierce). The dialysis cassette was placed in 2 liters of a solution of 1.55 g/l histidine, 9 g/l, pH 6.0 and dialyzed for 24 hours under light protection and stirring (150 rpm, magnetic stirrer). Subsequently, the dialysis buffer was replaced by 2 liters of fresh dialysis buffer (1.55 g/l histidine, 9 g/l NaCl, pH 6.0) and this procedure was repeated three more times for approximately 12 hours each. The resulting content of the dialysis cassette is referred to as HSA dialyzed or “HSA-dial.” The concentration was determined as described below.

Preparation of HSA-EDTA-dialyzed. 18 ml of HSA 25% (CSL Behring) was mixed with 2 ml of 1 M Na₂EDTA, pH 8.0. The obtained solution was stirred for approximately 6 hours. Then, about 15 ml of the solution was transferred to a dialysis cassette (Slide-A-Lyzer™, 10 kDa MWCO, 12-30 ml, Thermo Scientific/Pierce). The dialysis cassette was placed in 2 liters of a solution of 1.55 g/l histidine, 9 g/l NaCl, 37.2 g/l Na₂EDTA, pH 7.0 and dialyzed for 24 hours under light protection and stirring (150 rpm, magnetic stirrer).

Next, the dialysis buffer was replaced by 2 liters of 1.55 g/l histidine, 9 g/l NaCl, pH 7.0, followed by three cycles of dialyzing for approximately 12 hours and changing the dialysis buffer by 2 liters of fresh dialysis buffer (first buffer change by His/NaCl, pH 6.5 and second and third buffer changes by His/NaCl, pH 6.0). The resulting content of the dialysis cassette is the EDTA-dialyzed HSA material (or “HSA-EDTA-dial.”).

The HSA concentration after dialysis is generally significantly lower than the concentration of the starting HSA solution (25% w/v) due to the volume increase during dialysis. Since no significant losses are expected due to the high protein concentration, the absolute amount of HSA was considered unchanged and the concentration was calculated based on the volume change as known to a skilled person. The HSA concentration after dialysis was determined to be about 8.1% w/V.

Preparation of placebo bulk solutions. To prepare the formulations shown in Tables 6 and 7 but without the active agent (BoNT) (referred to herein as “placebo bulk solutions”), the starting materials HSA, NaCl, and histidine were weighed and dissolved in water (Milli-Q). 50 mM stock solutions of tryptophan (Trp) and N-acetyl-tryptophan (N-AcTrp) were prepared (10.212 g/l and 12.313 g/l, respectively, in warm water) and dosed according to the desired final Trp and N-AcTrp concentration. Finally, the pH was adjusted to 6.0 by adding 1 M NaOH or 1 M HCl, made up to the final volume with water, and the solution was sterile filtered through a 0.22 μm filter.

Preparation of BoNT pre-dilution. 13.44 mg of NT101 was weighed into a sterile 50 ml low protein binding tube and mixed with 20.123 g of the sterile filtered placebo bulk solution of formulation 7 to obtain a pre-dilution containing 25,000 U/ml BONT.

Preparation of samples. 20.0 μl of the BoNT pre-dilution was placed in sterile 15 ml low protein binding tube and 9.98 ml of the respective placebo bulk solution was added. The solution was gently inverted overhead 10 times to mix. Then, 3×1 ml of each solution was placed in 1.5 ml low binding tubes and these were exposed to 250 W/m² for 7 hours. The rest of each sample solution was covered with aluminum foil and bulk stored at 4° C. until measurement (“dark control” samples).

### Example 5

### Comparison of the Light Stability of Different Liquid BONT Formulations

The botulinum toxin activity of samples of formulations I to IV in Table 6 that were either exposed to light for 7 hours at 250 W/m² or stored in the dark for 7 hours was measured using the CBA (multiple measurements). The results are shown in Table 8 as percent activity of the light exposed sample to the dark stored control sample.

TABLE 8
Comparison of the light stability of
different liquid BoNT formulations

| | | Relative BoNT |
|---|---|---|
| BoNT formulation | | activity (% of dark |
| No. | HSA | control sample) |
| I | HSA-untreated | 46 |
| II | HSA-dial. | 56 |
| III | HSA untreated + | 80 |
| | EDTA | |
| IV | HSA-EDTA-dial. | 84 |

As can be seen from Table 8, dialysis of HSA somewhat increases the stability of BoNT to light exposure (see formulation II vs. formulation I). Addition of EDTA to the untreated HSA has a significantly greater positive effect (see formulation III). However, the addition of EDTA does not provide a complete protection against the destabilizing effect of light. The best light stability is achieved by pre-incubation with EDTA and subsequent dialysis against an EDTA-free histidine buffer (see formulation IV). This indicates that the observed light stability might not be due to metal ions alone but due to other unknown compounds.

### Example 6

### Light Stability of Liquid BONT Formulations in the Presence and Absence of Tryptophan and N-Acetyl-Tryptophan

The botulinum toxin activity of samples of the formulations 1 to 7 in Table 7 that were either exposed to light for 7 hours at 250 W/m² or stored in the dark for 7 hours was measured using the CBA (multiple measurements). The results are shown in Table 9 as percent activity of the light exposed sample to the dark stored control sample.

TABLE 9
Comparison of the light stability of liquid BoNT formulations
in the presence or absence of tryptophan or N-acetyl-tryptophan

| BoNT | | | Relative BoNT |
|---|---|---|---|
| formulation | | | activity (% of |
| | N-AcTrp | Trp | dark control |
| No. | (mM) | (mM) | sample) |
| 1 | 10 | — | 6 |
| 2 | 2 | — | 8 |
| 3 | 0.4 | — | 45 |
| 4 | 0.08 | — | 73 |
| 5 | — | 10 | 8 |
| 6 | — | 0.08 | 67 |
| 7 | — | — | 85 |

As can be seen, both tryptophan and N-acetyl-tryptophan have a marked negative effect on the stability of the BoNT liquid formulations to light in a dose dependent manner. At the higher concentrations (10 mM and 2 mM, respectively), the light stability decreasing effect was so pronounced that the relative biological activity was exceptionally low (<10%). At a concentration of as low as 80 μM of Trp or N-AcTrp, there is a decrease in BoNT biological activity of about 10% after exposure to light compared to the Trp- and N-AcTrp-free control formulation (see formulations 4 and 6 vs. formulation 7).

The concentration of 80 μM N-acetyl-tryptophan corresponds to a dilution of the 25% w/v HSA starting material (containing 20 mM N-AcTrp) to 0.1% w/v or 1 mg/ml HSA. Since 1 mg/ml HSA is a value that falls within a concentration range that is typically used in botulinum toxin formulations, the use of such an HSA starting material results in N-acetyl-tryptophan concentrations in BoNT/A formulations having an increased photosensitivity. Hence, the concentration range in which tryptophan and N-acetyl-tryptophan exert their negative impact on the light stability is of great practical importance.

1.-15. (canceled)

16. A liquid formulation, comprising
(i) botulinum toxin and
(ii) human serum albumin,
wherein the liquid formulation contains Fe³⁺ ions in a concentration of less than 1 μM.

17. The liquid formulation of claim 16, wherein the liquid formulation contains Fe³⁺ ions in a concentration of less than 500 nM.

18. The liquid formulation of claim 16, wherein the liquid formulation contains Fe³⁺ ions in a concentration of less than 250 nM.

19. The liquid formulation of claim 16, wherein the liquid formulation is prepared by a method comprising the following steps:
(a) contacting human serum albumin with a chelating agent to obtain a mixture of human serum albumin and the chelating agent, and
(b) removing the chelating agent from the mixture.

20. The liquid formulation of claim 19, wherein, in step (a), a human serum albumin starting material is contacted with a chelating agent to obtain a mixture of the human serum albumin starting material and the chelating agent and, in step (b), the chelating agent is removed from the mixture to obtain a pre-treated human serum albumin material and, in a further step (c), botulinum toxin is mixed with the pre-treated human serum albumin material.

21. The liquid formulation of claim 19, wherein, in step (a), a liquid pre-formulation comprising botulinum toxin and human serum albumin is contacted with the chelating agent to obtain a mixture of the liquid pre-formulation and the chelating agent and, in step (b), the chelating agent is removed from the mixture to obtain the liquid formulation.

22. The liquid formulation of claim 16, wherein the human serum albumin is present in the liquid formulation in an amount of 0.001-1.00% w/v.

23. The liquid formulation of claim 16, wherein the liquid formulation further comprises:
(iii) a tonicity agent,
or wherein the liquid formulation further comprises:
(iii) a tonicity agent, wherein the tonicity agent is present in the liquid formulation in an amount of 0.01-2.0% w/v, or wherein the tonicity agent is sodium chloride, or wherein the tonicity agent is sodium chloride and is present in the liquid formulation in an amount of 0.01-2.0% w/v.

24. The liquid formulation of claim 16, wherein the liquid formulation further comprises:
(iv) a buffering agent.

25. The liquid formulation of claim 24, wherein the buffering agent is present in the liquid formulation in a concentration of 1-100 mM.

26. The liquid formulation of claim 24, wherein the buffering agent is an amino acid, phosphate, or a mixture thereof.

27. The liquid formulation of claim 24, wherein the buffering agent is histidine, phosphate, or a mixture thereof, and the histidine and phosphate are present in the liquid formulation in a concentration of 1-100 mM.

28. A method of treating a cosmetic condition, neuromuscular diseases, pain, sialorrhea, hyperhidrosis, urological disorders, and neurological disorders comprising the steps of: administering the liquid formulation of claim 16 to a patient in need thereof.

29. A liquid formulation, comprising
(i) botulinum toxin and
(ii) human serum albumin,
wherein the liquid formulation contains no or no more than 50 μM tryptophan and N-acetyl-tryptophan.

30. The liquid formulation of claim 29, wherein the liquid formulation contains Fe³⁺ ions in a concentration of less than 1000 nM.

31. A method for preparing a liquid formulation according to claim 29, wherein the method comprises the following steps:
purifying a human serum albumin starting material to obtain a purified human serum albumin material,
mixing the obtained purified human serum albumin with botulinum toxin and optionally further components to obtain the liquid formulation,
wherein the purifying step is carried out by subjecting the human serum albumin starting material or the liquid composition comprising botulinum toxin and human serum albumin to dialysis, diafiltration, ultrafiltration, ion exchange chromatography, affinity chromatography, hydrophobic interaction chromatography, field flow fractionation or precipitation.

32. The method of claim 31, wherein the chelating agent is selected from the group consisting of aminopolycarboxylic acids having three to six carboxylic acid functional groups, citrate, porphyrins, N,N,N′,N′-tetrakis(2-pyridinylmethyl)-1,2-ethanediamine (TPEN), triethylenetetramine (TETA), and mixtures thereof.

33. The method of claim 31, wherein the purifying step comprises:
contacting a human serum albumin starting material with a chelating agent to obtain a mixture of the human serum albumin starting material and the chelating agent, and
removing the chelating agent from the mixture to obtain a chelating agent-treated purified human serum albumin material,
wherein the step of contacting is carried out such that the concentration of the chelating agent in mixture is 1 mM to 500 mM, and/or the removal of the chelating agent is carried out by dialysis, crossflow filtration or ultrafiltration.

34. A method for preparing a liquid formulation according to claim 29, wherein the method comprises the following steps:
purifying a liquid composition comprising botulinum toxin and human serum albumin to obtain a purified liquid composition,
mixing the obtained purified liquid composition with further components to obtain the liquid formulation,
wherein the purifying step is carried out by subjecting the human serum albumin starting material or the liquid composition comprising botulinum toxin and human serum albumin to dialysis, diafiltration, ultrafiltration, ion exchange chromatography, affinity chromatography, hydrophobic interaction chromatography, field flow fractionation or precipitation.

35. The method of claim 34, wherein the purifying step comprises:
contacting a liquid composition comprising botulinum toxin and human serum albumin with the chelating agent to obtain a mixture of the liquid composition and the chelating agent, and
removing the chelating agent from the mixture to obtain a chelating agent-treated purified liquid formulation,
wherein the step of contacting is carried out such that the concentration of the chelating agent in mixture is 1 mM to 500 mM, and/or the removal of the chelating agent is carried out by dialysis, crossflow filtration or ultrafiltration.

36. The method of claim 34, wherein the chelating agent is selected from the group consisting of aminopolycarboxylic acids having three to six carboxylic acid functional groups, citrate, porphyrins, N,N,N′,N′-tetrakis(2-pyridinylmethyl)-1,2-ethanediamine (TPEN), triethylenetetramine (TETA), and mixtures thereof.

37. The method of claim 36, wherein the chelating agent is an aminopolycarboxylic acid of general formula (I):
(HO₂CCH₂)₂N—R—N(CH₂CO₂H)₂ (I),
wherein R comprises no carboxylic acid group or one or two carboxylic acid groups.

38. The method of claim 37, wherein the aminopolycarboxylic acid is selected from the group consisting of ethylenediaminetetraacetic acid (EDTA), ethylene glycol-bis(b-aminoethylether)-N,N,N′,N′-tetraacetic acid (EGTA), 1,2-bis(o-aminophenoxy) ethane-N,N,N′,N′-tetraacetic acid (BAPTA), diethylenetriaminepentaacetic acid (DTPA), triethylenetetraminehexaacetate (TTHA), and mixtures thereof.

39. The liquid formulation of claim 29, wherein the botulinum toxin is a botulinum neurotoxin of serotype A, free of complexing proteins, and is present at a concentration of 1-1000 U/ml.

40. A method of treating neuromuscular diseases, pain, sialorrhea, hyperhidrosis, urological disorders, and neurological disorders comprising the steps of: administering the liquid formulation of claim 29 to a patient in need thereof.

41. A method of treating a cosmetic condition comprising the steps of: administering the liquid formulation of claim 29 to a patient in need thereof.

42. A liquid formulation, comprising
(i) botulinum toxin and
(ii) human serum albumin,
wherein the liquid formulation is prepared by a method comprising the following steps:
(a) contacting human serum albumin with a chelating agent to obtain a mixture of human serum albumin and the chelating agent, and
(b) removing the chelating agent from the mixture.
